(19) United States
(12) Patent Application Publication
(10) Pub. No.: US 2025/0248948 A1
(43) Pub. Date: Aug. 7, 2025

(54) Transdermal and/or Topical, Pharmaceutical Formulations Comprising Cannabidiol and/or Tetrahydrocannabinol for the Treatment of Chronic Pain

(71) Applicant: Pike Therapeutics Inc., Vancouver (CA)

(72) Inventors: Fotios M. PLAKOGIANNIS, Whitestone, NY (US); Tamanna LATHER, Jersey City, NJ (US)

(21) Appl. No.: 19/087,734

(22) Filed: Mar. 24, 2025

Related U.S. Application Data
(63) Continuation of application No. 17/235,314, filed on Apr. 20, 2021, now abandoned.
(60) Provisional application No. 63/012,428, filed on Apr. 20, 2020.

(51) Int. Cl. A61K 9/70; A61K 9/00; A61K 31/05; A61K 31/352; A61K 45/06

(52) U.S. Cl. CPC A61K 9/7046; A61K 9/0014; A61K 31/05; A61K 31/352; A61K 45/06

(57) ABSTRACT

The present disclosure relates to the to the transdermal administration of cannabinoids, such as, CBD and/or THC, and derivatives of these compounds, for the treatment and/or prevention and/or control of chronic pain.

This application is a continuation application of U.S. Ser. No. 17/235,314 filed Apr. 20, 2021, which claims benefit of U.S. Ser. No. 63/012,428 filed Apr. 20, 2020, the entirety of which is incorporated herein by reference.

### SPECIFICATION

### Background of the Invention

Pain results from noxious stimulation of nerve endings. Nociceptive pain is caused by noxious stimulation of nociceptors that transmit impulses over intact neural pathways to the spinal neurons and then to the brain. Peripheral neuropathic pain is pain due to damage of the nerve endings, mostly found in the skin, especially in the epidermis. These damaged nerve endings can generate impulses in the absence of stimulation, can be hypersensitive to normal stimulation, and/or can be triggered by remaining local inflammatory stimulation. Even a very small number of damaged and overactive small nerve fibers in the epidermis are sufficient to trigger peripheral neuropathic pain. Neuropathic pain can be debilitating and can reduce quality of life of patients considerably. This pain may persist for months or years beyond the apparent healing of any damaged tissues.

Neuropathic pain has a local inflammatory component that results in sensitization of nerve fibers. Other intact nerve fibers, such as nociceptors being present up in the stratum granulosum, innervating the same region can also be sensitized and participate in clinical symptoms of neuropathic pain (e.g., hyperalgesia). This results in a situation of local neurogenic inflammation resulting in many different clinical features such as burning, freezing, electric shock, itch, tingling, pins and needles, hyperalgesia and allodynia (pain resulting from a non-painful stimulus such as a light touch or stroke).

Peripheral nerve damage leads to enhanced transmitter release within the spinal cord and can lead to central sensitization. Increased peripheral input through primary afferents is critically involved in central sensitization and the maintenance of neuropathic pain. Peripherally acting drugs, such as lidocaine 5% medicated patches and capsaicin 8% patches, have demonstrated the ability to reduce pain in neuropathic pain syndromes. However, lidocaine patches are not easy to apply, especially on the toes and by elderly, because the patch has to be cut, and many elderly cannot reach their toes properly. Application of capsaicin creams and patches quite often induce intolerable side effects, such as an increase of burning sensation, and often the treatment has to be combined with a local anesthetic to neutralize this side effect.

In chronic pain in general, for instance, oral analgesics such as acetaminophen, nonsteroidal anti-inflammatory drugs (NSAIDs) and opioids are part of guidelines aimed at to reduce the pain. Chronic use of such oral analgesics however, can induce serious and mortal side effects and/or detrimental drug-drug interactions.

Topical painkiller pharmaceutical compositions are also explored to help patients suffering from chronic pain. Two most commonly used topical compounds in neuropathic pain are capsaicin (vanilloid receptor agonist and counter-irritant) and lidocaine (membrane stabilizer), and both have clear drawbacks.

Clearly, there remains a pressing and long felt need in the art of developing treatment options for chronic pain in general and neuropathic pain, in particular, for the development of a novel and effective pharmaceutical composition for use in the treatment of chronic pain, the composition having reduced side effects in the patient.

Multiple Sclerosis (MS) is the most common chronic autoimmune disease of central nervous system (CNS). MS can be characterized by inflammation, demyelination and neurodegeneration, which is resulted due to invasion of autoreactive myelin-specific T lymphocytes in CNS. These T cells triggers an inflammatory response including release of proinflammatory cytokines such as tumor necrosis factor (TNF) alpha, and Interferon (INF), addition of inflammatory cells, persistent activation of macrophages resulting in oligodendrocyte death and further demyelination. MS is classified in four major forms: 1) relapsing-remitting MS (RRMS), 2) Secondary progressive MS (SPMS), 3) Primary progressive MS (PPMS) and 4) Progressive-relapsing MS (PRMS). 85% or MS patients comes under RRMS group¹⁻⁵.

It is hard to find the cure for MS due to highly heterogeneous and unpredictable course of neurological deficits. Although several immunomodulatory and immunosuppressive agents such as IFN-beta, glatiramer acetate, dimethyl fumarate, mitoxantrone, teriflunomide, cladribine, fingolimod, Siponimod and ozanimod, natalizumab, alemtuzumab, ocrelizumab, showed success in slowing disease progression and decreasing the relapse rate. The clinical efficacy and risk benefits ratio of all above drugs are very low, and the more effective drugs have a higher risk of serious adverse reactions^{6,7}.

The other process for managing MS is use of wide array of pharmacological and non-pharmacological approaches designed to minimizing disease impact while maximizing quality of life. Among pharmacological treatment for the symptomatic management of MS, cannabis and its derivatives, such as delta-9-THC and cannabidiol (CBD) are increasingly recognized as effective to treat spasticity and pain. Currently, THC:CBD (1:1) ratio-called “Sativex” marketed in more than 25 countries (except USA) for treating spasticity related to MS. Furthermore, epidemiologic studies show that MS patients increasingly use cannabis preparation for a range of symptoms associated with MS symptoms, including sleep disturbance, pain, anxiety, spasticity and even depression. According to previous research, cannabis is used by 20-60% people to treat MS and related disease conditions^{5,8,9,10}.

Cannabis (marijuana) is a schedule-I drug in USA. Cannabis is a flowering plant which contains more than 400 phytonutrient (micronutrient). More than 100 different types of terpenoids, essential oils, antioxidants and cannabinoids have been extracted from the plant. Cannabinoids have immunomodulatory and immunosuppressive properties, suggesting these drugs as potential therapeutics in chronic inflammatory disease. Furthermore, cannabinoids receptors have been recently proposed as therapeutic targets for autoimmune disease including MS. The cannabis preparations can also be useful for chronic inflammatory conditions such as inflammatory bowel disease, rheumatoid arthritis, neurodegenerative disorders, and even in acute inflammation due to SARS-COV-2 infections¹¹⁻¹⁵.

From all of the phytochemicals, only tetrahydrocannabinol (THC) showed significant psychoactive effect. A number of research papers have been published on THC due to its psychoactive and therapeutic effects. Apart from THC, several other constituents have been studied, which also showed some therapeutic effect without psychoactive effect such as cannabidiol (CBD), cannbinol (CBN), cannabichromene (CBC), cannabigerol (CBG), tetrahydrocannbivarin (THCV), delta 9-tetrahydrocannbinol (delta9THC) and many more. It has been showed that cannabis and its derivatives can be used for the treatment of pain, type-2 related metabolic disorder, decrease intraocular pressure, Dravet syndrome, Lennox-Gastaut Syndrome (LGS), epilepsy, nausea, pain and wasting associated with AIDS, arthritis and rheumatism, migraines, muscle spasticity associated with multiple sclerosis and paralysis, alcohol and narcotics withdrawal, stress and depression, asthma, fibromyalgia, inflammatory pain, and pain and/or inflammation associated with chemotherapy, act as an antimicrobial. FDA approved Marinol and Syndros contains delta 9-THC, which currently used in treatment of nausea, vomiting, and anorexia associated with chemotherapy treatments. In clinical studies Sativex (cannabinoid extract oromucosal spray containing THC and CBD) has shown improvements in neuropathic pain and sleep quality. Currently, Sativex is available as an oromucosal spary, which delivers 2.7 mg THC and 2.5 mg CBD per spray. The current dosage regimen is to spray the formulation in mouth for 8-10 times a day. According to patent, EP1361864B9, the inventor made an argument that the oral cannabis delivery will metabolize 90% of the dose. Furthermore, they also suggested that the mucous membrane of the buccal cavity, under the tongue and the nasopharynx are not metabolizing cannabis and delivering them directly into the blood stream by avoiding first pass metabolism. During Phase-I clinical study, it was found that sublingual and buccal application is only 18% and 11% higher than the oral administration. This study concluded that the oromucosal spray will increase the bioavailability of cannabinoids from 6% to 8-10%. There is a lot of cannabinoids which are metabolizing through first pass metabolism. Furthermore, the application of oromucosal spary need to be standardized with relation to food intake in order to minimize the variability of bioavailability in the individual patients. (www.medicines.org.uk/emc/product/602/smpc#gref) In other words, the variability in the bioavailability through the oromucosal spary is very high. (https://pubmed.ncbi.nlm.nih.gov/23052407/. Furthermore, in April 2016 FDA gave orphan drug designation to cannabidiol for the treatment of Tuberous Sclerosis Complex (TSC), Dravet Syndrome and Lennox-Gastaut Syndrome. Cannabidiol is an orally effective treatment for pain and inflammation (Costa, B. The non-psychoactive cannabis constituent cannabidiol is an orally effective therapeutic agent in rat chronic inflammatory and neuropathic pain. European Journal of Pharmacology. Volume 556, Issues 1-3, 5 Feb. 2007, Pages 75-83).

Transdermal delivery of CBD/THC has a therapeutic potential for the management of MS. Currently, Sativex is given 8-10 sprays/day which is equivalent to 20 mg/day for CBD and THC individually. Upon consideration of bioavailability, the transdermal dose will be 2 mg/day.

The required flux for transdermal dose would be:

$$\begin{matrix}Required Flux = 2000 ug / 24 Hr / 50 \\ = 1.7 ug / sqcm / hr\end{matrix}$$

Furthermore, side effects related to oral delivery can be avoided using transdermal route. Furthermore, the peak and valley in the plasma concentration due to oral administration can be avoided by delivering the drug molecule constantly at predetermined input rate using transdermal dosage forms.

There are numerous patents available on cannabidiol, but the utility of those patents is not evaluated. One of the examples is the U.S. Pat. No. 9,375,417B2. According to patent' 5417, the inventors provided examples, but they failed to provide any in-vitro or in-vivo data for those examples. Due to lack of these data, the utility of patent is unfeasible.

Another example of these patents is U.S. Pat. No. 6,328,992B1. This patent provides all the examples for reservoir and adhesive matrix patches. All these examples contain mixture of cannabinoids (such as delta-8-THC, delta-9-THC, cannabidiol and cannabinol) instead of cannabidiol only. The THC is psychoactive agent and addictive substance. So, the utility of patent is problematic.

The current disclosure is addressed all the above drawbacks and provide patent which can have a real-world utility. Furthermore, current invention uses a synthetic version of cannabidiol which is manufactured in more controlled environment than the botanical source of the same. This could be another reason; synthetic version of THC/cannabidiol can provide more permeability as compared to adulterated version of it. Moreover, current invention is developing transdermal matrix patches which can deliver synthetic cannabidiol for 1 day, and/or 2-days, and/or 3-days, and/or 4 days, and/or 5 days, and/or 6 days, and/or 7 days, and/or upto 15 days.

There is a need for an improved drug delivery system of CBD and/or THC which can overcome the drawbacks associated with oral routes. Transdermal and/or topical delivery of CBD and/or THC, the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, solid solution thereof, polymorphs thereof, stereoisomers thereof, coated form thereof, ion-pairs thereof, solution thereof in solvents alone or in combinations thereof can address the challenges associated with oral drug delivery, and are useful as treatment, prevention and/or control of, for example, chronic pain or MS.

All references cited herein are incorporated herein by reference in their entireties.

### BRIEF SUMMARY OF THE INVENTION

The disclosure provides compositions and methods for the treatment and/or prevention and/or control of, for example, chronic pain, using transdermal drug delivery. In Transdermal drug delivery, a transdermal patch or transdermal composition is applied topically to the skin surface. Throughout the duration of topical application of a transdermal patch or transdermal composition drug is continuously released and delivered through the intact skin (via transcellular, intercellular and transappendageal routes) to achieve systemic effect. Therefore, once applied transdermal composition or transdermal patch can deliver drug into systemic circulation throughout the day or even for more than one day depending on the duration of its application which can be even up to a week and even up to fifteen days.

Transdermal delivery can reduce the dosing frequency of, for example, CBD and/or THC which is currently administered several times a day. Through transdermal delivery, transdermal compositions or transdermal formulations or transdermal patch of, for example, CBD and/or THC, can be applied topically to skin thereby delivering the drug throughout the duration of topical application. Depending on the requirement, the duration of topical application can be once in a day, once in two days, once in three days, once in four days, once in five days, once in a week, once in fifteen days. Therefore, transdermal delivery can overcome the multiple dose regimen of oral delivery by reducing the dosing frequency.

Moreover, in transdermal drug delivery the drug is delivered slowly and continuously throughout the duration of topical application hence there are no peaks and troughs in drug plasma concentration which are associated with multiple dose administration in a day. Therefore, by transdermal delivery of, for example, CBD and/or THC, patients can have the therapeutic effect of the drug for extended period of time without drastic changes in drug plasma concentration.

Furthermore, transdermal delivery is easy, noninvasive and convenient. Administration of a transdermal patch or transdermal composition does not require medical supervision as patients can topically apply the transdermal patch or transdermal composition themselves. Therefore, transdermal delivery can overcome the drawbacks of injections which are often painful and requires medical supervision.

With respect to CBD and/or THC it is expected that interpatient variability in pharmacologic response will be less with transdermal delivery as drug plasma concentration can be controlled by controlling the rate of drug delivery from transdermal composition or transdermal patch. With transdermal delivery a small amount of CBD and/or THC can be delivered for longer duration than oral administration. Transdermal formulations, for example, of CBD and/or THC also provide more abuse deterrence than immediate release dosage forms. Moreover, in case of any adverse effect, side effect or emergency transdermal delivery gives the liberty to terminate the therapy anytime by taking off the transdermal patch or transdermal composition from skin.

As per above stated reasons for the treatment and/or prevention and/or control of chronic pain, transdermal delivery can provide patient friendly, simplified and convenient therapeutic regimen over traditional delivery systems. Transdermal delivery can reduce the dosing frequency of CBD and/or THC. Depending on the necessity, dosing frequency can be once in a day, once in two days, once in three days, once in four days, once in five days, once in six days, once in a week, once in ten days. Through transdermal administration of drug combination, two or more drugs can be delivered simultaneously. Depending on the necessity, dosing frequency of transdermal patch or transdermal composition containing drug combination can be once in a day, once in two days, once in three days, once in four days, once in five days, once in six days, once in a week once in ten days. It would be a great addition to the patient compliance.

The disclosure provides a transdermal and/or topical pharmaceutical composition comprising: about 0.1% to about 20% of an active agent selected from the group consisting of cannabidiol (CBD), free base forms thereof, salts thereof, isomers thereof, amorphous forms thereof, derivatives thereof, and combinations thereof; about 0.1% to about 20% of an active agent selected from the group consisting of tetrahydrocannabinol (THC), free base forms thereof, salts thereof, isomers thereof, amorphous forms thereof, derivatives thereof, and combinations thereof; about 10% to about 50% of at least one solvent; about 10% to about 50% of at least surfactant; optionally, about 3% to about 15% of at least one permeation enhancer; optionally, about 5% to about 20% of an adhesive and/or polymer. The disclosure provides a transdermal and/or topical pharmaceutical composition wherein the THC is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, polymorph thereof, solid solution thereof, coated form thereof, stereoisomers thereof, solid solution thereof, ion-pair thereof, solution thereof, powder form thereof, liquid form thereof, alone or combinations thereof. The disclosure provides a transdermal and/or topical pharmaceutical composition wherein the CBD is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, polymorph thereof, solid solution thereof, coated form thereof, ion-pairs thereof, stereoisomers thereof, solid solution thereof, solution thereof, powder form thereof, liquid form thereof, alone or combinations thereof. The disclosure provides a transdermal and/or topical pharmaceutical composition comprising one or more active agent selected from the group consisting of tetrahydrocannabinol (THC), cannabidiol (CBD), the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, polymorph thereof, solid solution thereof, coated form thereof, and combinations thereof, in a dosage form for transdermal delivery. The disclosure provides a transdermal and/or topical pharmaceutical composition formulated as transdermal liquid formulation, transdermal semisolid formulation, transdermal gel formulation, or transdermal polymer matrix formulation, transdermal adhesive matrix formulation, transdermal film forming gel, transdermal film forming spray formulation, or transdermal drug-in-adhesive matrix formulation. The disclosure provides a transdermal and/or topical pharmaceutical composition formulated as a topical liquid formulation, topical semisolid formulation, topical gel formulation, topical polymer matrix formulation, topical adhesive matrix formulation, topical film forming gel formulation, or topical film forming spray formulation. The disclosure provides a transdermal and/or topical pharmaceutical composition further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, tackifier, diluent, bulking agent, solubilizers, suspending agents, dispersing agents, stabilizers, plasticizers, surfactants, antioxidants, oxidants, and combinations thereof. The disclosure provides a transdermal and/or topical pharmaceutical composition further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, solubilizers, suspending agents, dispersing agents, stabilizers, plasticizers, tackifiers, diluents, bulking agents, surfactants, antioxidants, oxidants, and combinations thereof in the range of 0.1%-99.5% w/w or w/v. The disclosure provides a pharmaceutical composition which is formulated as a transdermal patch. The disclosure provides a pharmaceutical composition which is formulated as metered dose transdermal gel, metered dose transdermal spray, a film forming gel, a film forming spray, or a meter-dose aerosol. The disclosure provides a pharmaceutical composition which is formulated as a topical patch. The disclosure provides a pharmaceutical composition which is formulated as metered dose gel, metered dose spray, gel, cream, solution, emulsion, liquid compositions, semisolid compositions, or film forming formulations. The disclosure provides a pharmaceutical composition formulated as a transdermal patch, wherein the transdermal patch is selected from the group such as to reservoir patch, a microreservoir patch, a matrix patch, a drug in adhesive patch, a pressure sensitive adhesive patch, extended-release transdermal film a liquid reservoir system, a microreservoir patch, a mucoadhesive patch, and combinations thereof. The disclosure provides a pharmaceutical composition formulated as a topical patch, wherein the topical patch is selected from the group such as to reservoir patch, a microreservoir patch, a matrix patch, a drug in adhesive patch, a pressure sensitive adhesive patch, extended-release transdermal film a liquid reservoir system, a microreservoir patch, a mucoadhesive patch, a micro-dosing patch, and combinations thereof. The disclosure provides a pharmaceutical composition indicated for the treatment and/or prevention and/or control of chronic pain in a patient. The disclosure provides a pharmaceutical composition indicated for the treatment and/or prevention and/or control of multiple sclerosis. The disclosure provides a pharmaceutical composition which is formulated as a transdermal formulation which can be administered in a dosage regimen selected from the group consisting of once daily, twice daily, three times a day, once in 1-8 hrs, once in 1-24 hrs, once in two days, once in three days, once in four days, once in five days, once in six days, once in a week, once in a 8 to about 13 days, once in two weeks, and once in 15 days to about 30 days. The disclosure provides a pharmaceutical composition which is formulated as a topical formulation which can be administered in a dosage regimen selected from the group consisting of once daily, twice daily, three times a day, four times a day, five times a day, six times a day, once in 1-8 hrs, once in 1-24 hrs, once in two days, once in three days, once in four days, once in five days, once in six days, once in a week, once in a 8 to about 13 days, once in two weeks, and once in 15 days to about 30 days. The disclosure provides a pharmaceutical composition formulated as microneedles. The disclosure provides a pharmaceutical composition wherein said tetrahydrocannabinol (THC), cannabidiol (CBD), the free base thereof, salts thereof, isomers thereof, amorphous forms thereof, polymorphs forms thereof, stereoisomers thereof, ion-pairs thereof, coated forms thereof, crystalline forms thereof, co-crystalline forms thereof, prodrugs thereof, analogs thereof, derivatives thereof, synthetic forms thereof, biosynthetic forms thereof, active metabolites thereof, and combinations thereof is produced by a synthetic route. The disclosure provides a pharmaceutical composition co-administered with at least one additional an active agent selected from the group consisting of: medications administered for treatment and/or management and/or prevention and/or control of symptoms associated with neuropathic pain, peripheral neuropathic pain, inflammatory pain, musculoskeletal pain, pain due to muscle spasms, pain due to increased muscle tone, osteoarthritic pain, muscular headache, tension-type headache, migraine, cluster headache, atypical facial pain, referred pain, vulvodynia, proctodynia, and any combination thereof. The disclosure provides a pharmaceutical composition further comprising at least one additional active agent selected from the group consisting of THC, CBD, antidepressant drug, NSAIDS, anticonvulsants drug, corticosteroid drug, pain relievers, lidocaine, menthol, capsaicin, methyl salicylate, lidocaine, capsaicin, Tricyclic Antidepressants, amitriptyline, imipramine, nortriptyline, desipramine, doxepin, SNRIs and SSRIs, duloxetine, venlafaxine, fluoxetine, milnacipran, diclofenac, aspirin, naproxen, ibuprofen, ketoprofen, celecoxib, meloxicam, acetaminophen, cox-2 inhibitors, celecoxib, anticonvulsants, carbamazepine, gabapentin, lamotrigine, pregabalin, oxcarbazepine, lamotrigine, valproic acid, menthol, camphor, methyl salicylate, salicylates, corticosteroid drugs, triamcinolone, methylprednisolone, cortisone, prednisone, dexamethasone, and opioids. The disclosure provides a method for the treatment and/or prevention and/or control of chronic pain in a patient comprising: selecting a patient in need of treatment and/or prevention and/or control of chronic pain; topically applying the pharmaceutical composition as disclosed herein, thereby treating, preventing and/or controlling chronic pain in the patient. The disclosure provides a method wherein the chronic pain is selected from the group consisting of neuropathic pain, peripheral neuropathic pain, inflammatory pain, musculoskeletal pain, pain due to muscle spasms, pain due to increased muscle tone, osteoarthritic pain, muscular headache, tension-type headache, migraine, cluster headache, atypical facial pain, referred pain, vulvodynia, proctodynia, and any combination thereof. The disclosure provides a method wherein the topical application of a transdermal pharmaceutical composition is for the treatment and/or prevention and/or control of chronic pain in a patient, and wherein the transdermal patch is applied at a time period selected from the group consisting of once in a day, once in two days, once in three days, once in four days, once in five days, once in six days, once in a week, and once in ten days, and once in fifteen days.

The disclosure provides a method for the treatment and/or prevention and/or control of multiple sclerosis in a patient comprising: selecting a patient in need of treatment and/or prevention and/or control of multiple sclerosis; topically applying the pharmaceutical composition as disclosed herein, thereby treating, preventing and/or controlling multiple sclerosis in the patient. The disclosure provides a method further providing a constant rate of delivery of the active components of the transdermal patch over a time period selected from the group consisting of once in a day, once in two days, once in three days, once in four days, once in five days, once in six days, once in a week, once in ten days, and once in fifteen days. The disclosure provides a method further providing a steady absorption rates of the active components of the transdermal patch over a time period selected from the group consisting of once in a day, once in two days, once in three days, once in four days, once in five days, once in six days, once in a week, once in ten days, and once in fifteen days. The disclosure provides a method further achieving a constant blood serum levels of the active components of the transdermal patch over a time period selected from the group consisting of once in a day, once in two days, once in three days, once in four days, once in five days, once in six days, once in a week, once in ten days, and once in fifteen days. The disclosure provides a method further achieving a reduced variability in dosage of the active components of the transdermal patches over a time period selected from the group consisting of once in a day, once in two days, once in three days, once in four days, once in five days, once in six days, once in a week, once in ten days, and once in fifteen days. The disclosure provides a method further providing a plasma concentration of the active components of the transdermal patch in a therapeutic range about 0.01 ng/ml to about 500 ng/ml. The disclosure provides a method further providing a plasma concentration of the active components of the transdermal patch in a therapeutic range of about 0.1 ng/ml to about 300 ng/ml. The disclosure provides a method wherein the topical application of a topical pharmaceutical composition is for the treatment and/or prevention and/or control of chronic pain in a patient, and wherein the topical patch is applied at a time period selected from the group consisting of once daily, twice daily, three times a day, four times a day, five times a day, once in 1-8 hrs, once in 1-24 hrs, once in a day, once in two days, once in three days, once in four days, once in five days, once in six days, once in a week, and once in ten days.

The disclosure provides for the use of the compounds and compositions of the disclosure for the production of a medicament for preventing and/or treating the indications as set forth herein.

In accordance with a further embodiment, the present disclosure provides for the use of the compounds and pharmaceutical compositions described herein, in an amount effective for use in a medicament, and most preferably for use as a medicament for treating a disease or disorder, for example, as set forth herein, in a subject.

In accordance with yet another embodiment, the present disclosure provides a use of the pharmaceutical compositions described above, and at least one additional therapeutic agent, in an amount effective for use in a medicament, and most preferably for use as a medicament for treating a disease or disorder associated with disease, for example, as set forth herein, in a subject.

The disclosure provides a method for treating and/or preventing a disease or condition as set forth herein in a patient, wherein said method comprises: selecting a patient in need of treating and/or preventing said disease or condition as set forth herein; administering to the patient a composition of the disclosure in a therapeutically effective amount, thereby treating and/or preventing said disease in said patient.

### DETAILED DESCRIPTION OF THE INVENTION

It is to be understood that this invention is not limited to particular embodiments described, as such may, of course, vary. It is also to be understood that the terminology used herein is for the purpose of describing particular embodiments only, and is not intended to be limiting, since the scope of this invention will be limited only by the appended claims.

The detailed description of the invention is divided into various sections only for the reader's convenience and disclosure found in any section may be combined with that in another section. Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a compound” includes a plurality of compounds.

Unless defined otherwise, all technical and scientific terms used herein have the same meaning as commonly understood by one of ordinary skill in the art to which this invention belongs. As used herein the following terms have the following meanings.

The terms transdermal and topical are used interchangeably

The term “chronic pain” or “chronic pain states” as used herein, is defined as any pain lasting longer than, for example, about 6 to about 12 weeks.

The term “neuropathic pain” as used herein has its conventional meaning and here means a pain arising as a direct or indirect consequence of a lesion or disease affecting the somatosensory system (central and/or peripheral). Neuropathic pain as used herein, includes all types of neuropathic pain, such as peripheral neuropathy caused by diabetes type 1 or 2, induced by various noxious substances such as alcohol, due to various deficiencies such as vitamin B1, B6 and/or B12 deficiency, various intoxications, such as hypervitaminosis B6, hypothyroidism, chemotherapeutic compound (e.g., paclitaxel or other taxane derivative, vincristine or other vinca alkaloids, cisplatin or other platinum derivate), drug-induced neuropathy, compounds for the treatment of infectious diseases (e.g., streptomycin, didanosine or zalcitabine), or any other chemically toxic compound. Other peripheral neuropathies include the following: trigeminal neuralgia, post-herpetic neuralgia, intercostal neuralgia, entrapment neuropathy (e.g., carpal tunnel syndrome, tarsal tunnel syndrome, abdominal cutaneous nerve entrapment syndrome), small fiber neuropathy, hereditary motor and sensory neuropathies, chronic inflammatory demyelinating polyneuropathy, sciatic pain chronic idiopathic sensory neuropathy, infectious disease conditions such as post-polio syndrome, AIDS or HIV-associated, Lyme-associated, Sjogren-associated, lymphomatous neuropathy, myelomatous neuropathy, carcinomatous neuropathy, acute pan autonomic neuropathy, vasculitic/ischaemic neuropathy and other mono- and polyneuropathies. Furthermore, under the term “neuropathic pain” also the following is included: complex regional pain syndrome type I and II (reflex sympathetic dystrophy), central neuropathic pain (e.g., thalamic neuropathy, spinal cord injury neuropathy, post stroke pain, multiple sclerosis neuropathy, syringomyelia, spinal cord tumors), phantom limb pain, restless genital syndrome (pain), post-surgical scar pain including cardiac surgery and mastectomy.

The term “inflammatory pain” as used herein has its conventional meaning and here means a pain that arises from inflammation that may be caused but by not limited to trauma, burns, extreme cold, fractures, (osteo) arthritis, rheumatoid arthritis, chronic strains, surgery, infection and autoimmune diseases excessive stretching, infections and vasoconstriction. Multiple inflammatory mediators can directly affect nociceptors or may sensitize them to touch or movement, even some distance from the inflammatory field.

The term “musculoskeletal pain” as used herein has its conventional meaning and here means a pain that affects the muscles, ligaments, tendons, bones, joints and/or soft tissues that are part of the musculoskeletal system. Musculoskeletal pain as used herein, includes all types of pain due to damage of muscle tissue as a result of wear and tear of daily activities. Trauma to an area (jerking movements, auto accidents, falls, sport injuries, fractures, sprains, strains dislocations, and direct blows to the muscle) also can cause musculoskeletal pain. Other causes of musculoskeletal pain include postural strain, repetitive movements, overuse, and prolonged immobilization, misuse of muscles, fibromyalgia, lumbar pain, pain due to increased muscle tone, and tendinitis due to overuse.

The term “treatment” as used herein has its conventional meaning and is here to be considered in its broadest context. The term “treatment” is intended to encompass topical administration of active compounds, i.e., active pharmaceutical ingredients e.g., in a pharmaceutical composition, according to the disclosure, with the aim to alleviate an undesired condition, and therapeutic administration to eliminate or reduce the extent or symptoms of the condition. Treatment does not necessarily imply that a subject is treated until total recovery.

The term “analgesic” or “analgesics” as used herein has its conventional meaning and here refers to compounds, agents, drugs or substances that reduce pain in its broadest context.

The term “co-analgesic” or “co-analgesics” as used herein has its conventional meaning and here refers to compounds, agents, drugs or substances whose primary indication is for a purpose other than pain relief, which compounds demonstrate analgesic activity.

The term “reinstating analgesic effects” as used herein has its regular scientific meaning and is here referring to the capability (of a compound or of a composition) of reinstating an analgesic effect of at least one analgesic compound or at least one co-analgesic compound, when decreasing analgesic effect occurs after repeated use of a topical formulation containing at least one analgesic or co-analgesic compound.

The term “effect booster” or “co-analgesic effect booster” or “therapeutic effect booster” or “booster effect” or “synergistic effect” as used herein has its conventional meaning and here means the enhancement of a therapeutic effect induced by a co-analgesic compound (“co-analgesic”) leading to 1) intensified therapeutic effects of an active pharmaceutical ingredient with the purpose of alleviating neuropathic pain, inflammatory pain, musculoskeletal pain, pain due to muscle spasms, and/or other chronic pain states, 2) a faster onset of pain relieving effect, 3) a longer duration of analgesia, and/or 4) reinstating analgesic effects, when decreasing analgesic effect occurs after repeated use of a topical pharmaceutical composition containing at least one analgesic compound (“analgesic”) or co-analgesic compound.

The term “topical formulation” as used herein has its conventional meaning and here refers to a formulation that may be applied to skin or mucosa with the aim that a therapeutically active compound penetrates in and/or through the skin, e.g., a topical pharmaceutical composition of the disclosure, e.g., a pharmaceutical composition provided as a topical cream.

As used herein, the term “transdermal delivery” means delivery of drug into systemic circulation through the skin.

As used herein, the term “topical delivery” means delivery of drug to skin for local effect

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is a human. As used herein, the term “agent” refers to any molecule, compound, methodology and/or substance for use in the prevention, treatment, management and/or diagnosis of a disease or condition. As used herein, the term “effective amount” refers to the amount of a therapy that is sufficient to result in the prevention of the development, recurrence, or onset of a disease or condition, and one or more symptoms thereof, to enhance or improve the prophylactic effect(s) of another therapy, reduce the severity, the duration of a disease or condition, ameliorate one or more symptoms of a disease or condition, prevent the advancement of a disease or condition, cause regression of a disease or condition, and/or enhance or improve the therapeutic effect(s) of another therapy.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the term “therapeutic agent” refers to any molecule, compound, and/or substance that is used for treating and/or managing a disease or disorder.

As used herein, the terms “therapies” and “therapy” can refer to any method(s), composition(s), and/or agent(s) that can be used in the prevention, treatment and/or management of a disease or condition, or one or more symptoms thereof. In certain embodiments, the terms “therapy” and “therapies” refer to small molecule therapy.

The term “derivative” or “derivatized” as used herein includes, for example, chemical modification of a compound of the disclosure, or extracted from botanical sources or pharmaceutically acceptable salts thereof or mixtures thereof. That is, a “derivative” may be a functional equivalent of a compound of the disclosure, which is capable of inducing the improved pharmacological functional activity in a given subject.

As used herein, the terms “composition” and “formulation” are used interchangeably.

### Active Agent

The term “active ingredient” refers to an agent, active ingredient compound or other substance, or compositions and mixture thereof that provide some pharmacological, often beneficial, effect. Reference to a specific active ingredient shall include where appropriate the active ingredient and it's pharmaceutically acceptable salts. The disclosure provides for, for example, transdermal formulations and/or topical formulations comprising one or more of the following active agents: Cannabinoids are a group of 21-carbon-containing terpenophenolic compounds produced by Cannabis species. Cannabinoids may also be synthetically produced. The term “cannabinoid” refers hereinafter to a class of diverse chemical compounds that act on cannabinoid receptors on cells that repress neurotransmitter release in the brain. These receptor proteins include the endocannabinoids (produced naturally in the body by humans and animals), the phytocannabinoids (found in cannabis and some other plants), and synthetic cannabinoids. Lipophilic cannabinoids are generally grouped as endocannabinoids (most typically as mammalian endocannabinoids); phytocannabinoids, from plant sources; and synthetic cannabinoids. Such cannabinoids are also often classified into the following subclasses: Cannabigerols (CBG); Cannabichromenes (CBC); Cannabidiol (CBD; CBDL); Tetrahydrocannabinol (THC); Cannabinol (CBN); Cannabicyclol (CBL); Cannabielsoin (CBE); and, Cannabitriol (CBT).

Cannabidiol IUPAC Name 2-[(1R,6R)-6-isopropenyl-3-methylcyclohex-2-en-1-yl]-5-pentylbenzene-1,3-diol Chemical Formula: C₂₁H₃₀O₂ Molecular weight: 314.46 dalton Chemical structure is shown below as formula I

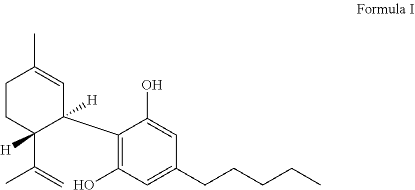 [SMILES: C=C(C)[C@@H]1CCC(C)=C[C@H]1c1c(O)cc(CCCCC)cc1O]

Tetrahydrocannbinol (THC) IUPAC Name (−)-(6aR,10aR)-6,6,9-Trimethyl-3-pentyl-6a,7,8,10a-tetrahydro-6H-benzo[c]chromen-1-ol Chemical Formula: C₂₁H₃₀O₂ Molecular weight: 314.47 dalton.

Chemical structure is shown below as formula II

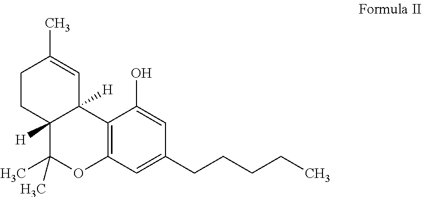 [SMILES: CCCCCc1cc(O)c2c(c1)OC(C)(C)[C@@H]1CCC(C)=C[C@@H]21]

As used herein, the word cannabis refers to all pharmaceutically acceptable forms of cannabis and its derivatives either alone or in combinations thereof, for example, in following forms but not limited to such as free base or salts or isomers or amorphous or crystalline or co crystalline or solid solution or prodrugs or analogs or derivatives or metabolites or polymorphs or its stereoisomer or coated form or ion-pairs. For example, cannabidiol's free base or its salts or its isomers or its amorphous form or its crystalline form or its co crystalline form or its solid solution or its prodrugs or its analogs or its derivatives or synthetic forms or its polymorphs or its stereoisomer or its ion-pairs. The compound may be in the form of, for example, a pharmaceutically acceptable salt, such as an acid addition salt or a base salt, or a solvate thereof, including a hydrate thereof. Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts. Suitable base salts are formed from bases which form non-toxic salts and examples are the sodium, potassium, aluminium, calcium, magnesium, zinc and diethanolamine salts.

As used herein, the term “cannabidiol” includes the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, solid solution thereof, polymorph thereof, stereoisomers thereof, powder form thereof, liquid form thereof, ion-pairs thereof, solution of cannabidiol in solvents such as but not limited to methanol, etc. alone or in combinations thereof.

As used herein, the term “THC” includes the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, solid solution thereof, powder form thereof, liquid form thereof, ion-pairs thereof, polymorph thereof, stereoisomers thereof, solution of THC in solvents such as but not limited to methanol, heptane, etc. alone or in combinations thereof.

As used herein, synthetic cannabinoids include at least the following: AM-087 is an analgesic drug that is a cannabinoid agonist derivative of Δ8THC substituted on the 3-position side chain and a potent CB1 agonist; AM-251 is an inverse agonist at the CB1 cannabinoid receptor with close structural similarity to SR141716A (rimonabant), both of which are biarylpyrazole cannabinoid receptor antagonists as well as μ-opioid receptor antagonist; Methanandamide (AM-356) is a stable chiral analog of anandamide and acts on the cannabinoid receptors with a Ki of 17.9 nM at CB1 and 868 nM at CB2; AM-374—palmitylsulfonyl fluoride; AM-381—stearylsulfonyl fluoride; known AM404, also as N-arachidonoylaminophenol, is an active metabolite of paracetamol (acetaminophen) thought to induce its analgesic action through its activity on the endocannabinoid, COX, and TRPV systems, all of which are present in pain and thermoregulatory pathways; AM-411 is an analgesic that is a cannabinoid agonist; AM-411 is a potent and fairly selective CB1 full agonist and produces similar effects to other cannabinoid agonists such as analgesia, sedation, and anxiolysis; AM-630 (6-lodopravadoline) acts as a potent and selective inverse agonist for the cannabinoid receptor CB2, selectivity over CB1 where it acts as a weak partial agonist; AM-661—1-(N-methyl-2-piperidine)methyl-2-methyl-3-(2-iodo)benzoylindole; JWH-018 (1-pentyl-3-(1-naphthoyl) indole) or AM-678 is an analgesic chemical from the naphthoylindole family that acts as a full agonist at both the CB1 and CB2 cannabinoid receptors, with some selectivity for CB2; AM-679 acts as a moderately potent agonist for the cannabinoid receptors; AM-694 (1-(5-fluoropentyl)-3-(2-iodobenzoyl) indole) acts as a potent and selective agonist for the cannabinoid receptor CB1; AM-735—3-bornyl-Δ8-THC, a mixed CB1/CB2 agonist; AM-855 is an analgesic cannabinoid agonist at both CB1 and CB2 with moderate selectivity for CB1; AM-881—a chlorine-substituted stereoisomer of anandamide whose Ki=5.3 nM at CB1 and 95 nM at CB2; AM-883 an allyl-substituted stereoisomer of anandamide whose Ki-9.9 nM at CB1 and 226 nM at CB2; AM-905 is an analgesic cannabinoid which acts as a potent and reasonably selective agonist for the CB1 cannabinoid receptor; AM-906 is an analgesic drug which is a cannabinoid agonist and is a potent and selective agonist for the CB1 cannabinoid receptor; AM-919 is an analgesic cannabinoid receptor agonist, potent with respect to both CB1 and CB2; AM-926—a potent agonist at both CB1 and CB2 with moderate selectivity for CB1; AM-938 is an analgesic drug which is a cannabinoid receptor agonist and while it is still a potent agonist at both CB1 and CB2, it is reasonably selective for CB2; AM-1116—a dimethylated stereoisomer of anandamide; AM-1172—an endocannabinoid analog specifically designed to be a potent and selective inhibitor of AEA uptake that is resistant to FAAH hydrolysis; AM-1220 is a potent and moderately selective agonist for the cannabinoid receptor CB1; AM-1221 acts as a potent and selective agonist for the cannabinoid receptor CB2; AM-1235 (1-(5-fluoropentyl)-3-(naphthalen-1-oyl)-6-nitroindole) acts as a potent and reasonably selective agonist for the cannabinoid receptor CB1; AM-1241 (1-(methylpiperidin-2-ylmethyl)-3-(2-iodo-5-nitrobenzoyl) indole) is a potent and selective agonist for the cannabinoid receptor CB2, with analgesic effects in mammals, particularly against “atypical” pain such as hyperalgesia and allodynia, and has also shown efficacy in the treatment of amyotrophic lateral sclerosis in mammalian models; AM-1248 acts as a moderately potent agonist for both the cannabinoid receptors CB1 and CB2; AM-1710—a CB2 selective cannabilactone with 54× selectivity over CB1; AM-1714 acts as a reasonably selective agonist of the peripheral cannabinoid receptor CB2 and has both analgesic and anti-allodynia effects; AM-2201 (1-(5-fluoropentyl)-3-(1-naphthoyl) indole) acts as a potent but nonselective full agonist for the cannabinoid receptor; AM-2212—a potent agonist at both CB1 and CB2; AM-2213—a potent agonist at both CB1 and CB2; AM-2232 (1-(4-cyanobutyl)-3-(naphthalen-1-oyl) indole) acts as a potent but unselective agonist for the cannabinoid receptors CB1 and CB2; AM-2233 acts as a highly potent full agonist for the cannabinoid receptors CB1 and CB2 and has been found to fully substitute for THC in certain mammalian studies, with a potency lower than that of JWH-018 but higher than WIN 55,212-2; AM-2389 acts as a potent and reasonably selective agonist for the CB1 receptor; AM-3102—an analog of oleoylethanolamide, (the endogenous agonist for proliferator-activated receptor α (PPARα)) it acts as a weak cannabinoid agonist at CB1 and at CB2; AM-4030 an analgesic which is potent agonist at both CB1 and CB2, but also reasonably selective for CB1; AM-4054 is a potent but slow-onset agonist with CB1 affinity and selectivity CB1 over CB2; AM-4113—a CB1 selective neutral antagonist; AM-6545 acts as a peripherally selective silent antagonist for the CB1 and was developed for the treatment of obesity; JWH-007—an analgesic which acts as a cannabinoid agonist at both the CB1 receptor and CB2 receptors, with some selectivity for CB2, JWH-007 is an analgesic which acts as a cannabinoid agonist at both the CB1 and CB2 receptors; JWH-015 acts as a subtype-selective cannabinoid agonist which binds almost 28× more strongly to CB2 than CB1, and has been shown to have immunomodulatory effects, and may be useful in the treatment of pain and inflammation; JWH-018 an analgesic which acts as a full agonist at both the CB1 and CB2 cannabinoid receptors and produces effects similar to those of THC; JWH-019—an agonist at both CB1 and CB2 receptors and is an analgesic from the naphthoylindole family that acts as a cannabinoid agonist at both the CB1 and CB2 receptors; JWH-030—an analgesic which is a partial agonist at CB1 receptors; JWH-047—a potent and selective agonist for the CB2 receptor, JWH-048—a potent and selective agonist for the CB2 receptor, JWH-051—an analgesic with a high affinity for the CB1 receptor, but is a much stronger agonist for CB2, JWH-057—a 1-deoxy analog of Δ8-THC that has very high affinity for the CB2 receptor, but also has high affinity for the CB1 receptor; JWH-073—an analgesic which acts as a cannabinoid agonist at both the CB1 and CB2 receptors. It is somewhat selective for the CB1 subtype; JWH-081—an analgesic which acts as an agonist at both the cannabinoid CB1 AND CB2 receptors; JWH-098—a potent and fairly selective CB2 agonist; JWH-116—a CB1 ligand; JWH-120—a potent and 173-fold selective CB2 agonist; JWH-122—a potent and fairly selective CB1 agonist; JWH-133—a potent and highly selective CB2 receptor agonist; 1JWH-139—3-(1,1-dimethylpropyl)-6,6,9-trimethyl-6a,7,10,10a-tetrahydro-6H-benzo[c]chromene; JWH-147—an analgesic from the naphthoylpyrrole family, which acts as a cannabinoid agonist at both the CB1 and CB2 receptors; JWH-148—a moderately selective ligand for the CB2 receptor, with more than 8 times selectivity over the CB1 subtype; JWH-149—a potent and fairly selective CB2 agonist; JWH-161—a CB1 ligand; JWH-164—a potent cannabinoid agonist; JWH-166—a potent and highly selective CB2 agonist; JWH-167—a weak cannabinoid agonist from the phenylacetylindole family; JWH-171—an analgesic which acts as a cannabinoid receptor agonist; JWH-175-(1-pentylindol-3-yl) naphthalen-1-ylmethane, 22 nM at CB1, JWH-176—1-([(1E)-3-pentylinden-1-ylidine]methyl) naphthalene; JWH-181—a potent cannabinoid agonist; JWH-182—a potent cannabinoid agonist with some selectivity for CB1; JWH-184—1-pentyl-1H-indol-3-yl-(4-methyl-1-naphthyl) methane; JWH-185—1-pentyl-1H-indol-3-yl-(4-methoxy-1-naphthyl) methane; JWH-192—(1-(2-morpholin-4-ylethyl) indol-3-yl)-4-methylnaphthalen-1-ylmethane; JWH-193—(1-(2-morpholin-4-ylethyl) indol-3-yl)-4-methylnaphthalen-1-ylmethanone; JWH-194—2-methyl-1-pentyl-1H-indol-3-yl-(4-methyl-1-naphthyl) methane; JWH-195—(1-(2-morpholin-4-ylethyl) indol-3-yl)-naphthalen-1-ylmethane; JWH-196—2-methyl-3-(1-naphthalenylmethyl)-1-pentyl-1H-Indole; JWH-197—2-methyl-1-pentyl-1H-indol-3-yl-(4-methoxy-1-naphthyl) methane; JWH-198—(1-(2-morpholin-4-ylethyl) indol-3-yl)-4-methoxynaphthalen-1-ylmethanone; JWH-199—(1-(2-morpholin-4-ylethyl) indol-3-yl)-4-methoxynaphthalen-1-ylmethane; JWH-200—an analgesic from the aminoalkylindole family, which acts as a cannabinoid receptor agonist; JWH-203—an analgesic from the phenylacetylindole family, which acts as a cannabinoid agonist with approximately equal affinity at both the CB1 and CB2 receptors; JWH-205—142-methyl-1-pentylindol-3-yl)-2-phenylethanone; JWH-210—an analgesic chemical from the naphthoylindole family, which acts as a potent cannabinoid agonist at both the CB1 and CB2 receptors; JWH-213—a potent and fairly selective CB2 agonist; JWH-229—1-methoxy-3-(1′,1′-dimethylhexyl)-Δ8-THC, a dibenzopyran cannabinoid which is a potent CB2 agonist; JWH-234—a cannabinoid agonist with selectivity for CB2; JWH-250—an analgesic from the phenylacetylindole family, which acts as a cannabinoid agonist at both the CB1 and CB2 receptors; JWH-251—(1-pentyl-3-(2-methylphenylacetyl) indole); JWH-258—a potent and mildly selective CB1 agonist; JWH-302—(1-pentyl-3-(3-methoxyphenylacetyl) indole); JWH-307—an analgesic from the naphthoylpyrrole family, which acts as a cannabinoid agonist at both the CB1 and CB2 receptors that is somewhat selective for the CB2 subtype; JWH-350—a 11-nor-1-methoxy-3-(1′,1′-dimethylheptyl)-9α-hydroxyhexahydrocannabinol has a 33-fold selectivity for the CB2 receptor and high CB2receptor affinity with little affinity for the CB1 receptor; JWH-359—a dibenzopyran cannabinoid that is a potent and selective CB2 receptor agonist; JWH-387-1—pentyl-3-(4-bromo-1-naphthoyl) indole, an analgesic from the naphthoylindole family, which acts as a potent cannabinoid agonist at both receptors CB1 and CB2; JWH-398—an analgesic chemical from the naphthoylindole family, which acts as a potent cannabinoid agonist at both receptors with a Ki of 2.3 nM at CB1 and 2.8 nM at CB2; JWH-424—a potent and moderately selective CB2 agonist with a Ki of 5.44 nM at CB2 and 20.9 nM at CB1; HU-210 is a cannabinoid that is 100 to 800 times more potent than natural THC from cannabis and has an extended duration of action and is a potent analgesic with many of the same effects as natural THC; Ajulemic acid (AB-III-56, HU-239, IP-751, CPL 7075, CT-3, Resunab) is a cannabinoid derivative of the non-psychoactive THC metabolite 11-nor-9-carboxy-THC that shows useful analgesic and anti-inflammatory effects without causing a subjective “high”. It is being developed for the treatment of neuropathic pain and inflammatory conditions such as arthritis and for the treatment of orphan life-threatening inflammatory diseases; HU-243 (AM-4056) is a cannabinoid which is a potent agonist at both the CB1 and CB2 receptors; HU-308 acts as a cannabinoid agonist and is highly selective for the CB2 receptor subtype. It has analgesic effects, promotes proliferation of neural stem cells, and protects both liver and blood vessel tissues against oxidative stress via inhibition of TNF-α; HU-331 is a quinone anticarcinogenic synthesized from cannabidiol; HU-336 is a strongly antiangiogenic compound, it inhibits angiogenesis by directly inducing apoptosis of vascular endothelial cells without changing the expression of pro- and anti-angiogenic cytokines and their receptors; HU-345 (cannabinol quinone) is a drug that is able to inhibit aortic ring angiogenesis more potently than its parent compound cannabinol; CP 47,497 or (C7)-CP 47,497 is a cannabinoid receptor agonist drug.

The disclosure also provides methods for the biosynthesis of cannabinoids and for the use of a eukaryotic or prokaryotic expression system for the production of biosynthetic enzymes that can be used for the manufacture of cannabinoids and cannabinoid analogs. Yeast as well as eukaryotic and prokaryotic cells are suitable for the cloning and expression of the cannabinoid acid synthase enzymes and include without limitation E coli, yeast and baculovirus hosts. Thus, the present disclosure provides a method for the production of biosynthetic cannabinoids, such as for example THC and/or CBD, using cannabinoid acid synthase enzymes including, but not limited to, tetrahydrocannabinolic acid (THCA) synthase and cannabidiolic acid (CBDA) synthase. The disclosure further provides for the transdermal and/or topical compositions as disclosed herein comprising, for example, biosynthetic CBD, alone or in combination with other active agents.

According to certain embodiments, transdermal and/or topical compositions described herein are for the prevention and/or treatment of pain and/or inflammation. According to certain embodiments, transdermal and/or topical compositions described herein are for the reduction in severity of pain and/or inflammation.

According to certain embodiments described herein, pharmaceutical composition or transdermal formulation or topical formulation contains cannabidiol and/or THC—the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, polymorphs thereof, stereoisomers thereof, coated form thereof, solid solution thereof, ion-pairs thereof, solution thereof in solvents alone or in combinations thereof. More preferably transdermal and topical formulation may include cannabidiol, the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, polymorphs thereof, ion-pairs thereof, stereoisomers thereof, coated form thereof, solution of cannabidiol in methanol, alone or in combinations thereof. More preferably transdermal and topical formulation may include THC, the free base thereof, salts thereof, isomers thereof, amorphous forms thereof, crystalline forms thereof, co crystalline forms thereof, prodrugs thereof, analogs thereof, derivatives thereof, ion-pairs thereof, synthetic forms thereof, biosynthetic forms thereof, active metabolites thereof, polymorphs thereof, stereoisomers thereof, coated form thereof, solution of cannabidiol in methanol, alone or in combinations thereof.

As used herein, the word active agent refers to all pharmaceutically acceptable forms of the active agent and its derivatives either alone or in combinations thereof, for example, in following forms but not limited to such as free base or salts or isomers or amorphous or crystalline or co crystalline or solid solution or prodrugs or analogs or derivatives or metabolites polymorphs thereof, stereoisomers thereof, coated form thereof, ion-pairs thereof. For example, the active agent's free base or its salts or its isomers or its amorphous form or its crystalline form or its co crystalline form or its solid solution or its prodrugs or its analogs or its derivatives or synthetic forms polymorphs thereof, ion-pairs thereof, stereoisomers thereof, coated form thereof. The compound may be in the form of, for example, a pharmaceutically acceptable salt, such as an acid addition salt or a base salt, or a solvate thereof, including a hydrate thereof. Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts. Suitable base salts are formed from bases which form non-toxic salts and examples are the sodium, potassium, aluminium, calcium, magnesium, zinc and diethanolamine salts. The active ingredient(s) can be present in the form of a free base or in the form of pharmaceutically acceptable salts. Pharmaceutically acceptable salts forming part of this invention are intended to define but not limited to salts of the carboxylic acid moiety such as alkali metal salts like Li, Na and K salts; alkaline earth metal salts like Ca and Mg salts; salts of organic bases such as lysine, arginine, guanidine, diethanolamine, choline, and the like; ammonium or substituted ammonium salts and aluminium salts. Salts may be acid addition salts which defines but not limited to sulfates, nitrates, phosphates, perchlorates, borates, hydrohalides, acetates, tartrates, maleates, citrates, succinates, palmoates, methanesulfonates, benzoates, salicylates, hydroxynaphthoates, benzensulfonates, ascorbates, glycerophosphates, ketoglutarates and the like.

As used herein, the term active agent includes the free base thereof, salts thereof, isomers thereof, amorphous forms thereof, crystalline forms thereof, co crystalline forms thereof, prodrugs thereof, analogs thereof, derivatives thereof, synthetic forms thereof, polymorphs thereof, stereoisomers thereof, coated form thereof, ion-pairs thereof, alone or in combinations thereof. In certain embodiments the active agent is highly purified. In certain embodiments the active agent is present as a highly purified extract of active agent which comprises at least 90%, 91%, 92%, 93%, 94%, 95%, 96%, 97%, 98%, 99%, 99.5%, or 99.75% (w/w) of the formulation in certain embodiments, the dose of active agent is greater than, for example, about 0.01, 0.1, 0.25, 0.5, 1, 2, 3, 4, 5, 6, 7, 8, 9, 10, 15, 20, 25, 30, 35, 40, or 45 mg/kg/day. In certain embodiments, the dose of active agent is greater than, for example, about 0.01, 0.1, 0.25, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, or 275 mg/day. In exemplary embodiments, formulations of the disclosure may comprise active agent at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise active agent at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the active agent will represent approximately 1 wt % to 75 wt %, preferably 2 wt % to 30 wt %, more preferably 5 wt. % to 20 wt. % of the formulation.

As used herein, the term “pharmaceutically acceptable salts” includes acid addition salts or addition salts of free bases. The term “pharmaceutically acceptable salts” of the active agent within its scope all the possible isomers and their mixtures, and any pharmaceutically acceptable metabolite, bioprecursor and/or pro-drug, such as, for example, a compound which has a structural formula different from the one of the compounds of the disclosure, and yet is directly or indirectly converted in vivo into a compound of the disclosure, upon administration to a subject, such as a mammal, particularly a human being.

As used herein, the term “transdermal delivery” means delivery of drug into systemic circulation through the skin.

### Additional Active Agents

As used herein the term “combination administration” of a compound, therapeutic agent or known drug with the combination of the present invention means administration of the drug and the one or more compounds at such time that both the known drug and/or combination will have a therapeutic effect. In some cases this therapeutic effect will be synergistic. Such concomitant administration can involve concurrent (i.e. at the same time), prior, or subsequent administration of the drug with respect to the administration of the composition and/or combination of the present invention. A person of ordinary skill in the art would have no difficulty determining the appropriate timing, sequence and dosages of administration for particular drugs of the present invention.

Further, active ingredient(s), where applicable, may be present either in the form of one substantially optically pure enantiomer or as a mixture of enantiomers or polymorphs thereof.

The active ingredient(s) may comprise one or more of the following therapeutic classes but not limited to adrenergic agent; adrenocortical steroid; adrenocortical suppressant; aldosterone antagonist; amino acid; anabolic; analeptic; analgesic; anesthetic; anorectic; anti-acne agent; anti-adrenergic; anti-allergic; anti-amebic; anti-anemic; anti-anginal; anti-arthritic; anti-asthmatic; anti-atherosclerotic; antibacterial; anticholinergic; anticoagulant; anticonvulsant; antidepressant; antidiabetic; antidiarrheal; antidiuretic; anti-emetic; anti-epileptic; antifibrinolytic; antifungal; antihemorrhagic; antihistamine; antihyperlipidemia; antihypertensive; antihypotensive; anti-infective; anti-inflammatory; antimicrobial; antimigraine; antimitotic; antimycotic, antinauseant, antineoplastic, antineutropenic, antiparasitic; antiproliferative; antipsychotic; antirheumatic; antiseborrheic; antisecretory; antispasmodic; antithrombotic; anti-ulcerative; antiviral; appetite suppressant; blood glucose regulator; bone resorption inhibitor; bronchodilator; cardiovascular agent; cholinergic; depressant; diagnostic aid; diuretic; dopaminergic agent; estrogen receptor agonist; fibrinolytic; fluorescent agent; free oxygen radical scavenger; gastric acid supressant; gastrointestinal motility effector; glucocorticoid; hair growth stimulant; hemostatic; histamine H2 receptor antagonists; hormone; hypocholesterolemic; hypoglycemic; hypolipidemic; hypotensive; imaging agent; immunizing agent; immunomodulator; immunoregulator; immunostimulant; immunosuppressant; keratolytic; LHRH agonist; mood regulator; mucolytic; mydriatic; nasal decongestant; neuromuscular blocking agent; neuroprotective; NMDA antagonist; non-hormonal sterol derivative; plasminogen activator; platelet activating factor antagonist; platelet aggregation inhibitor; psychotropic; radioactive agent; scabicide; sclerosing agent; sedative; sedative-hypnotic; selective adenosine A1 antagonist; serotonin antagonist; serotonin inhibitor; serotonin receptor antagonist; steroid; thyroid hormone; thyroid inhibitor; thyromimetic; tranquilizer; amyotrophic lateral sclerosis agent; cerebral ischemia agent; Paget's disease agent; unstable angina agent; vasoconstrictor; vasodilator; wound healing agent; xanthine oxidase inhibitor.

Examples of active ingredients comprises, but is not limited to any of the following, for example, alone or in combination: Examples of active ingredients comprise drugs which are administered for the treatment and/or prevention and/or control and/or management of symptoms associated with neuropathic pain, peripheral neuropathic pain, inflammatory pain, musculoskeletal pain, pain due to muscle spasms, pain due to increased muscle tone, osteoarthritic pain, muscular headache, tension-type headache, migraine, cluster headache, atypical facial pain, referred pain, vulvodynia, proctodynia alone or in combinations thereof.

In one aspect examples of active ingredients comprise drugs such as but not limited to lidocaine, capsaicin, Tricyclic Antidepressants (not limited to such as amitriptyline, imipramine, nortriptyline, desipramine, doxepin, etc.), SNRIs and SSRIs (not limited to such as duloxetine, venlafaxine, fluoxetine, milnacipran etc.), NSAIDS (not limited to diclofenac, aspirin, naproxen, ibuprofen, ketoprofen, celecoxib, meloxicam, etc.), acetaminophen, cox-2 inhibitors (not limited to celecoxib, etc.), anticonvulsants (not limited to such as carbamazepine, gabapentin, lamotrigine, pregabalin, oxcarbazepine, lamotrigine, etc.), valproic acid, menthol, camphor, methyl salicylate, salicylates, corticosteroid drugs (not limited to such as triamcinolone, methylprednisolone, cortisone, prednisone, dexamethasone, etc), opioid, etc.

In one aspect transdermal delivery system and/or topical delivery system comprising drug combination of two or more drugs such as but not limited to THC, CBD, lidocaine, menthol, capsaicin, methyl salicylate, etc. Examples of drug combination of two or more drugs for transdermal delivery systems and/or topical delivery system includes such as but not limited to THC, CBD, lidocaine and combinations thereof, THC, CBD, menthol and combinations thereof, THC, CBD, capsaicin and combinations thereof, THC, CBD, methyl salicylate and combinations thereof, etc.

In another aspect transdermal delivery system and/or topical delivery system comprising drug combination of two or more drugs such as but not limited to THC, CBD, antidepressant drug, NSAIDS, anticonvulsants drug, corticosteroid drug, pain relievers, etc. Examples of drug combination of two or more drugs for transdermal delivery systems and/or topical delivery system includes such as but not limited to THC, CBD, antidepressant drug and combinations thereof, THC, CBD and anticonvulsant drug and combinations thereof, THC, CBD, corticosteroid drug and combinations thereof, THC, CBD, NSAID drug and combinations thereof, etc.

As indicated the pharmaceutical formulations as disclosed herein may comprise auxiliary excipients such as for example diluents, binders, lubricants, surfactants, disintegrants, plasticisers, tackifiers, opacifying agents, pigments, and such like. As will be appreciated by those skilled in the art, the exact choice of excipient and their relative amounts will depend to some extent on the final transdermal or topical dosage form.

### Pharmaceutical Compositions

According to certain embodiments described herein, pharmaceutical composition or transdermal formulation and/or topical formulation of contains active agents such as cannabinoids, and derivatives of these compounds. More preferably transdermal and/or topical formulation may include active agents such as CBD and/or THC, and derivatives of these compounds.

One embodiment of the present disclosure can be a transdermal drug delivery system which may include without any limitation to transdermal formulation, transdermal patches, microneedles, iontophoresis, metered dose transdermal spray, metered dose transdermal gel, transdermal aerosols, transdermal film forming formulations.

Transdermal formulation which includes liquids for example without any limitation like solutions, suspensions, dispersions, emulsion. Transdermal formulation includes semisolids for example without any limitations like gels, ointments, emulsions, creams, suspension, paste, lotion, balm. Liquid formulation and/or gel formulation incorporated in transdermal patch, metered dose transdermal system, sachet, etc. Transdermal formulations which includes matrix patches without any limitations like adhesive matrix patch, drug in adhesive matrix patch, non-adhesive matrix patch, a transdermal matrix formulation as drug in adhesive matrix patch is preferred. Other transdermal formulations include transdermal gel, transdermal meter dose spray, transdermal meter dose aerosols, transdermal film forming formulation, microneedles.

Without any limitation, transdermal patch may include all transdermal drug delivery systems stated in art preferably but not limited to reservoir patch, matrix patch, bilayer matrix patch, multilayer matrix patch, microreservoir patch, adhesive systems, transdermally applicable tape and other.

In certain embodiments of the present disclosure, a transdermal patch comprises transdermal formulation containing active agents such as CBD and/or THC, and derivatives of these compounds contained in a reservoir or a matrix, and an adhesive which allows the transdermal patch to adhere to the skin, allowing the passage of the active agents such as CBD and/or THC, and derivatives of these compounds from the transdermal patch through the skin of the patient. The transdermal delivery system can be occlusive, semi-occlusive or non-occlusive, and can be adhesive or non-adhesive.

The transdermal formulation comprising active agents such as CBD and/or THC, and derivatives of these compounds can be incorporated within the patch and patch can be applied topically to the skin surface. The patch can be left on the subject for any suitable period of time.

In some embodiments, the transdermal patches provide for a constant rate of delivery of the active components of the transdermal patch over a predetermined time period. In some embodiments, the predetermined time period is 24 hours, 48 hours, 72 hours, 96 hours, 120 hours, 144 hours, 7 days, 8 to 13 days, two weeks, or 15 days.

In yet further embodiments, the transdermal patches described herein provide a steady absorption rate of the active components of the transdermal patches by the patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the transdermal patches described herein provide a constant blood serum level of the active components of the transdermal patches in a patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the transdermal patches described herein provide a plasma concentration of the active components of the transdermal patches in a therapeutic range in a patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the transdermal patches described herein allow for reduced variability in dosage of active components in a patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the transdermal patches described herein provide a plasma concentration of the active components of the transdermal patches in a therapeutic range in a patient over a predetermined time. In exemplary embodiments as disclosed herein, the transdermal patch provides a blood serum level of active agent selected from without any limitation, of, for example, about 0.01 ng/ml, about 0.02 ng/mL, about 0.05 ng/ml, about 0.1 ng/mL, about 0.2 ng/mL, about 0.5 ng/mL, about 1 ng/mL, about 2 ng/ml, about 5 ng/ml, about 10 ng/ml, about 20 ng/ml, about 50 ng/mL, about 100 ng/mL, about 200 ng/mL, about 500 ng/ml, about 1 μg/mL mL, about 2 μg/mL, about 5 μg/mL, and ranges thereof. In one aspect, transdermal patch provides a blood serum level of active agent in the range of 0.01 ng/mL-400 ng/mL. In another aspect, transdermal patch provides a blood serum level of active agent in the range of 0.01 ng/mL-100 ng/mL. In yet another aspect transdermal patch provides a blood serum level of active agent in the range of from 0.01-1 ng/ml to 1-100 ng/ml to 100-500 ng/ml to 500-1000 ng/ml to 1000-5000 ng/ml.

The topical formulation stated in the art which include, for example without any limitation, semisolids such as ointment, cream, emulsion, micro emulsion, nano emulsion, paste, balms, gels, lotions, mousses. Liquids such as solutions, suspensions, micro suspension, nano suspension, dispersions, nano dispersion etc. Sprays, aerosols, magma, etc. The topical formulation comprising such as CBD and/or THC, and derivatives of these compounds can be topically applied to the skin surface for topical delivery of such CBD and/or THC, and derivatives of these compounds.

One embodiment of the present disclosure can be a topical drug delivery system which may include without any limitation to topical patches, topical formulation, metered dose topical spray, topical film forming formulation, topical drug-in-adhesive patches, topical matrix patches, topical aerosols, metered dose topical gel.

Topical formulation which includes liquids for example without any limitation like solutions, suspensions, dispersions, emulsion. Topical formulation includes semisolids for example without any limitations like gels, ointments, emulsions, creams, suspension, paste, lotion, balm. Liquid formulation and/or gel formulation incorporated in without any limitation to topical patch, metered dose topical system, sachet, etc. Topical formulations which includes polymer matrix patch without any limitations like adhesive matrix patch, non-adhesive matrix, drug-in-adhesive matrix patch, a topical matrix formulation as drug in adhesive matrix patch is preferred. Other topical formulations include such as but not limited to topical gel, metered dose topical spray, metered dose topical aerosols, topical film forming formulation.

Without any limitation, topical patch may include all topical drug delivery systems stated in art preferably but not limited to reservoir patch, matrix patch, bilayer matrix patch, multilayer matrix patch, microreservoir patch, adhesive systems, topically applicable tape and other.

In certain embodiments of the present disclosure, a topical patch comprises topical formulation containing active agents such as diclofenac and/or CBD and/or THC, and derivatives of these compounds contained in a reservoir or a matrix, and an adhesive which allows the topical patch to adhere to the skin, allowing the passage of the active agents such as diclofenac and/or CBD and/or THC, and derivatives of these compounds from the topical patch to the skin of the patient. The topical delivery system can be occlusive, semi-occlusive or non-occlusive, and can be adhesive or non-adhesive.

The topical formulation comprising active agents such as diclofenac and/or CBD and/or THC, and derivatives of these compounds can be incorporated within the patch and patch can be applied topically to the skin surface. The patch can be left on the subject for any suitable period of time.

The transdermal formulation and/or topical formulation of some embodiments of the present disclosure may include carriers or ingredients in effective amount either alone or in combinations thereof without any limitation to the following carriers or ingredients such as solvents, gelling agents, polymers, pressure sensitive adhesive polymers, adhesive polymers biodegradable polymers, penetration enhancers, emollients, skin irritation reducing agents, buffering agents, pH stabilizers, solubilizers, suspending agents, dispersing agents, stabilizers, plasticizers, tackifiers, surfactants, volatile chemicals, antioxidants, oxidants, chelating agents, complexing agents, diluents, bulking agents, excipients, material to prepare patch, material to prepare matrix patch, material to prepare reservoir patch etc.

Active agents may be dissolved, suspended, dispersed or uniformly mixed in the above stated single carrier, mixture of carriers and combinations of carrier. Any combination of two or more drugs such as such as CBD and/or THC, and derivatives of these compounds may be dissolved, suspended, dispersed or uniformly mixed in the above stated single carrier, mixture of carriers and combinations of carrier.

The desired optimum transdermal and/or topical formulation of such as CBD and/or THC, and derivatives of these compounds alone or in combinations thereof may comprise without any limitation to following carriers as stated from example 1 to example 12 either alone or in combinations thereof.

According to certain embodiments, transdermal and/or topical compositions described herein are for the treatment and/or prevention and/or control of, for example, chronic pain.

### Indications

One embodiment of the disclosure is the pharmaceutical composition for use in the treatment of chronic pain according to the disclosure, wherein the composition contains active agent as disclosed herein, and wherein the composition is administered every other day, daily, twice daily, three times daily or four times daily for a period of at least one day, at least one week, anytime between one week to one year, at least one year, or longer. One embodiment of the disclosure is the pharmaceutical composition for use in the treatment of chronic pain according to the disclosure, wherein the composition is administered every other day, daily, twice daily, three times daily or four times daily for a period of at least one day, at least one week, anytime between one week to one year, at least one year, or longer. This way, a continuous decrease of (peripheral) neuropathic pain, inflammatory pain, musculoskeletal pain, pain due to muscle spasms, and/or other chronic pain states is achieved upon administering the pharmaceutical composition of the disclosure to a patient suffering from chronic pain.

In one embodiment, the pharmaceutical composition for use in the treatment of chronic pain according to the disclosure is a pharmaceutical transdermal composition wherein the use is the transdermal use in the treatment of chronic pain according to the disclosure. In one embodiment, the pharmaceutical composition for use in the treatment of chronic pain according to the disclosure is a pharmaceutical topical composition wherein the use is the topical use in the treatment of chronic pain according to the disclosure. Here, topical composition will be topically applied to intact skin area experiencing pain in the treatment of chronic pain.

In one embodiment, the pharmaceutical composition for use in the treatment of chronic pain according to the disclosure is a pharmaceutical transdermal composition wherein the use is the transdermal use on intact skin of the treated person in the treatment of chronic pain according to the disclosure.

In one embodiment, the pharmaceutical composition for use in the treatment of chronic pain according to the disclosure, is a pharmaceutical transdermal composition wherein the use is the transdermal use on healthy intact skin of the treated person in the treatment of chronic pain according to the disclosure. Here, intact skin and healthy intact skin have their common scientific meaning and here refer to non-injured skin free of e.g., ulcers, wounds, lesions, cuts, and refer to skin comprising a closed outer layer of epidermis.

One embodiment of the disclosure is a pharmaceutical composition according to the disclosure or provided by the method of the disclosure, for use in the treatment of chronic pain according to the disclosure, wherein the chronic pain is neuropathic pain, peripheral neuropathic pain, inflammatory pain, musculoskeletal pain, pain due to muscle spasms, pain due to increased muscle tone, osteoarthritic pain, muscular headache, tension-type headache, migraine, cluster headache, atypical facial pain, referred pain, vulvodynia, proctodynia, or combinations thereof.

In one embodiment, the pharmaceutical composition for use in the treatment of chronic pain according to the disclosure is the pharmaceutical composition, wherein the chronic pain is peripheral neuropathic pain.

One embodiment of the disclosure is a pharmaceutical composition according to the disclosure or provided by the method of the disclosure, for use in the treatment of chronic pain according to the disclosure, wherein the chronic pain is neuropathic pain selected from peripheral neuropathy caused by diabetes type 1 or 2, or induced by a noxious substance such as alcohol, due to vitamin B1, B6 and/or B12 deficiency, hypervitaminosis B6, hypothyroidism, chemotherapeutic compound such as paclitaxel or a taxane derivative, vincristine or a vinca alkaloid, cisplatin or a platinum derivate, drug-induced neuropathy, a compound for the treatment of infectious disease such as streptomycin, didanosine or zalcitabine, a chemically toxic compound, trigeminal neuralgia, post-herpetic neuralgia, intercostal neuralgia, entrapment neuropathy such as carpal tunnel syndrome, tarsal tunnel syndrome, abdominal cutaneous nerve entrapment syndrome, sciatic pain chronic idiopathic sensory neuropathy, small fiber neuropathy, hereditary motor and sensory neuropathies, chronic inflammatory demyelinating polyneuropathy, infectious disease conditions such as post-polio syndrome, AIDS or HIV-associated, Lyme-associated, Sjogren-associated, lymphomatous neuropathy, myelomatous neuropathy, carcinomatous neuropathy, acute pan autonomic neuropathy, vasculitic/ischaemic neuropathy and a mono- and polyneuropathy, complex regional pain syndrome type I and II (reflex sympathetic dystrophy), central neuropathic pain such as thalamic neuropathy, spinal cord injury neuropathy, post stroke pain, multiple sclerosis, multiple sclerosis neuropathy, syringomyelia, a spinal cord tumor, phantom limb pain, restless genital syndrome with pain, post-surgical scar pain including scar pain after cardiac surgery and mastectomy.

One embodiment of the disclosure is a pharmaceutical composition according to the disclosure or provided by the method of the disclosure, for use in the treatment of chronic pain according to the disclosure, wherein the dosing frequency of the pharmaceutical composition is between once every other day and eight times daily, preferably six, five, four, three, two or one times daily.

One embodiment of the disclosure is the pharmaceutical composition for use in the treatment of chronic pain according to the disclosure, wherein the pharmaceutical composition is administered during a period of at least one day, preferably at least one week, more preferably at least one month, most preferably at least one year, preferably the pharmaceutical composition is administered for one to ten years, more preferably the pharmaceutical composition is administered chronically. It is to be understood that it is part of the disclosure that the pharmaceutical composition for use in the treatment of chronic pain according to the disclosure is administered to patients suffering from chronic pain for the rest of their lifespan. This way, the chronic pain is at least less intense and preferably patients are relieved from the chronic pain to a large extent or even completely.

The invention will be illustrated in more detail with reference to the following Examples, but it should be understood that the present invention is not deemed to be limited thereto.

### EXAMPLES

### Example 1

The transdermal formulation and/or topical formulation of the disclosure may comprise solvents known to those skilled in the art either alone or in combinations thereof without any limitation to following like alcohol C₁-C₂₀ such as but not limited to (methanol, ethanol, isopropyl alcohol, butanol, propanol etc.), polyhydric alcohols, glycols such as but not limited to (propylene glycol, polyethylene glycol, dipropylene glycol, hexylene glycol, butyene glycol, glycerine etc.), derivative of glycols, pyrrolidone such as but not limited to (N methyl 2-pyrrolidone, 2-pyrrolidone etc.), sulfoxides such as but not limited to (dimethyl sulfoxide, decymethylsulfoxide etc), dimethylisosorbide, mineral oils, vegetable oils, sesame oil water, polar solvents, semi polar solvents, non polar solvents, volatile chemicals which can be used to make matrix patch such as but not limited to (ethanol, propanol, ethyl acetate, acetone, methanol, dichloromethane, chloroform, toluene, IPA, hexane), acids such as but not limited to acetic acid, lactic acid, levulinic acid, bases and others, pentane, dimethylformamide, butane, lipids. More preferably in the range of 0.01%-95% w/w or w/v. In exemplary embodiments, formulations of the disclosure may comprise solvents at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80%, and about 95% of the formulation. In exemplary embodiments, formulations of the disclosure may comprise solvents at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the solvents will represent approximately 1 wt % to 75 wt %, preferably 2 wt % to 30 wt %, more preferably 5 wt. % to 20 wt. % of the formulation.

### Example 2

The transdermal formulation and/or topical formulation of the disclosure may comprise gelling agents and/or thickening and/or suspending agents and/or polymers and/or adhesive polymers and/or pressure sensitive adhesive polymers known to those skilled in the art either alone or in combinations thereof without any limitation to following like natural polymers, polysaccharides and its derivatives such as but not limited to (agar, alginic acid and derivatives, cassia tora, collagen, gelatin, gellum gum, guar gum, pectin, potassium, or sodium carageenan, tragacanth, xantham, gum copal, chitosan, resin etc.), semisynthetic polymers and its derivatives such as without any limitation to cellulose and its derivatives (methylcellulose, ethyl cellulose, carboxymethyl cellulose, hydroxylpropyl cellulose, hydroxylpropylmethyl cellulose etc.), synthetic polymers and its derivatives such as without any limitation to carboxyvinyl polymers or carbomers (carbopol 940, carbopol 934, carbopol 971p NF), polyethylene, and its copolymers etc, clays such as but not limited to (silicates, bentonite), silicon dioxide, polyvinyl alcohol, acrylic polymers (eudragit), acrylic acid esters, polyacrylate copolymers, polyacrylamide, polyvinyl pyrrolidone homopolymer and polyvinyl pyrrolidone copolymers such as but not limited to (PVP, Kollidon 30, poloxamer), isobutylene, ethyl vinyl acetate copolymers, natural rubber, synthetic rubber, pressure sensitive adhesives such as silicone polymers such as but not limited to (bio psa 4302, bio-psa 4202 etc.,), acrylic pressure sensitive adhesives such as but not limited to (duro-tak 87-2156, duro-tak 387-2287, duro-tak 87-9301, duro-tak 387-2051 etc.), polyisobutylene such as but not limited to (polyisobutylene low molecular weight, plyisobutylene medium molecular weight, polyisobutylene 35000 mw, etc), acrylic copolymers, rubber based adhesives, hot melt adhesives, styrene-butadiene copolymers, bentonite, all water and/or organic solvent swellable polymers, etc. In exemplary embodiments, formulations of the disclosure may comprise gelling agents and/or thickening and/or suspending agents and/or polymers and/or adhesive polymers and/or pressure sensitive adhesive polymers at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80%, and about 85%, and about 90% of the formulation. In exemplary embodiments, formulations of the disclosure may comprise gelling agents and/or thickening and/or suspending agents and/or polymers and/or adhesive polymers and/or pressure sensitive adhesive polymers at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the gelling agents and/or thickening and/or suspending agents and/or polymers and/or adhesive polymer and/or pressure sensitive adhesive polymers will represent approximately 1 wt % to 75 wt %, preferably 2 wt % to 30 wt %, more preferably 5 wt. % to 20 wt. % of the formulation, and more preferably in the range of 0.1% 80% w/w or w/v.

### Example 3

The transdermal formulation and/or topical formulation of the disclosure may comprise permeation enhancers known to those skilled in the art either alone or in combination thereof without any limitation to the following, such as sulfoxides, and similar chemicals such as but dimethylacetamide, dimethylformamide, not limited to (dimethylsulfoxide, decymethylsulfoxide, dimethylisosorbide etc), azone, pyrrolidones such as but not limited to (N-methyl-2-pyrrolidone, 2-pyrrolidon etc.), esters, fatty acid esters such as but not limited to (propylene glycol monolaurate, butyl ethanoate, ethyl ethanoate, isopropyl myristate, isopropyl palmitate, methyl ethanoate, lauryl lactate, ethyl oleate decyl oleate, glycerol monooleate, glycerol monolaurate, lauryl laurate etc.), fatty acids such as but not limited to (capric acid, caprylic acid, lauric acid, oleic acid, myristic acid, linoleic acid, stearic acid, palmitic acid etc.), alcohols, fatty alcohols and glycols such as but not limited to (oleyl alcohol, nathanol, dodecanol, propylene glycol, glycerol etc.), ethers alcohol such as but not limited to (diethylene glycol monoethyl ether), urea, triglycerides such as but not limited to triacetin, polyoxyethylene fatty alcohol ethers, polyoxyethylene fatty acid esters, esters of fatty alcohols, essential oils, surfactant type enhancers such as but not limited to (brij, sodium lauryl sulfate, tween, polysorbate), terpene, terpenoids and all penetration or permeation enhancers referred in the book “Percutaneous Penetration Enhancers” (Eric W. Smith, Howard I. Maibach, 2005. November, CRC press). In exemplary embodiments, formulations of the disclosure may comprise permeation enhancers at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise permeation enhancers at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the permeation enhancers will represent approximately 1 wt % to 75 wt %, preferably 2 wt % to 30 wt %, more preferably 5 wt. % to 20 wt. % of the formulation, and more preferably in the range of 0.01%-95% w/w or w/v.

### Example 4

The transdermal formulation and/or topical formulation of the disclosure may comprise plasticizers known to those skilled in the art either alone or in combination thereof without any limitation to following like glycerol and its esters, phosphate esters, glycol derivatives, sugar alcohols, sebacic acid esters, citric acid esters, tartaric acid esters, adipate, phthalic acid esters, triacetin, oleic acid esters and all the plasticizers which can be used in transdermal drug delivery system referred in the book “Handbook of Plasticizers” (George Wypych, 2004, Chem Tec Publishing). In exemplary embodiments, formulations of the disclosure may comprise plasticizers at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise plasticizers at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the plasticizers will represent approximately 1 wt % to 75 wt %, preferably 2 wt % to 30 wt %, more preferably 5 wt. % to 20 wt. % of the formulation. More preferably in the range of 0.01%-95% w/w or w/v.

### Example 5

The transdermal formulation and/or topical formulation of the disclosure may comprise emollients, humectants, skin irritation reducing agents and similar compounds or chemicals known to those skilled in the art either alone or in combinations thereof without any limitation to following like petrolatum, lanolin, mineral oil, dimethicone, zinc oxide, glycerin, propylene glycol and others. More preferably in the range of 0.01%-95% w/w or w/v. In exemplary embodiments, formulations of the disclosure may comprise emollients, humectants, skin irritation reducing agents and similar compounds at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise emollients, humectants, skin irritation reducing agents and similar compounds at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the emollients, humectants, skin irritation reducing agents and similar compounds will represent approximately 1 wt % to 75 wt %, preferably 2 wt % to 30 wt %, more preferably 5 wt. % to 20 wt. % of the formulation, and more preferably in the range of 0.01%-95% w/w or w/v.

### Example 6

The transdermal formulation and/or topical formulation of the disclosure may comprise solubilizers, surfactants, emulsifying agents, dispersing agents and similar compounds or chemicals known to those skilled in the art either alone or in combination thereof without any limitation to following like polysorbate such as but not limited to (polysorbate 20, polysorbate 40, polysorbate 60, polysorbate 80 etc.), span such as but not limited to (span 80, span 20 etc.), surfactants such as (anionic, cationic, nonionic and amphoteric), propylene glycol monocaprylate type I, propylene glycol monocaprylate type II, propylene glycol dicaprylate, medium chain triglycerides, propylene glycol monolaurate type II, linoleoyl polyoxyl-6 glycerides, oleoyl-polyoxyl-6-glycerides, lauroyl polyoxyl-6-gylcerides, polyglyceryl-3-dioleate, diethylene glycol monoethyl ether, propylene glycol monolaurate type I, polyglyceryl-3-dioleate, caprylocaproyl polyoxyl—8 glycerides etc, cyclodextrins and others. More preferably in the range of 0.01% 95% w/w or w/v. In exemplary embodiments, formulations of the disclosure may comprise solubilizers, surfactants, emulsifying agents, dispersing agents and similar compounds at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise solubilizers, surfactants, emulsifying agents, dispersing agents and similar compounds at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the solubilizers, surfactants, emulsifying agents, dispersing agents and similar compounds will represent approximately 1 wt % to 75 wt %, preferably 2 wt % to 30 wt %, more preferably 5 wt. % to 20 wt. % of the formulation, and more preferably in the range of 0.01% 95% w/w or w/V.

### Example 7

Different techniques and ingredients can be used to increase the stability and/or solubility of the active agents in formulation such as without any limitation to coating, encapsulation, microencapsulation, nanoencapsulation, lyophilization, chelating agents, complexing agents, etc.

### Example 8

The transdermal formulation and/or topical formulation of the disclosure may comprise auxiliary pH buffering agents and pHI stabilizers and similar compounds known to those skilled in the art which helps to maintain the appropriate pH of formulation preferably in the range of 4.0-8.0 either alone or in combination thereof without any limitation to following such as phosphate buffer, acetate buffer, citrate buffer, etc., acids such as but not limited to (carboxylic acids, inorganic acids, sulfonic acids, vinylogous carboxylic acids and others), base such as but not limited to (sodium hydroxide, potassium hydroxide, ammonium hydroxide, triethylamine, sodium carbonate, sodium bicarbonate) etc. More preferably in the range of 0.01%-30% w/w or w/v. In exemplary embodiments, formulations of the disclosure may comprise pH buffering agents and pH stabilizers and similar compounds at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise pH buffering agents and pH stabilizers and similar compounds at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the pH buffering agents and pH stabilizers and similar compounds will represent approximately 1 wt % to 75 wt %, preferably 2 wt % to 30 wt %, more preferably 5 wt. % to 20 wt. % of the formulation, and more preferably in the range of 0.01%-30% w/w or w/v.

### Example 9

The transdermal formulation and/or topical formulation of the disclosure may comprise antioxidants such as but not limited to (sodium metabisulfite, citric acid, ascorbic acid, BHA, BHT), oxidizing agents, stabilizers, discoloring agents, preservatives and similar compounds or chemicals known to those skilled in the art which helps to get a stable formulation can be used either alone or in combination thereof without any limitation. More preferably in the range of 0.01%-50% w/w or w/v. In exemplary embodiments, formulations of the disclosure may comprise antioxidants at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise antioxidants at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the antioxidants will represent approximately 1 wt % to 75 wt %, preferably 2 wt % to 30 wt %, more preferably 5 wt. % to 20 wt. % of the formulation, and more preferably in the range of 0.01%-50% w/w or w/v.

### Example 10

The transdermal formulation and/or topical formulation of the disclosure may be formulated in ointment and/or cream base and/or gel and/or film forming formulation and/or transdermal matrix formulation and/or drug-in-adhesive matrix patch and/or matrix patch known to those skilled in the art.

### Example 11

Materials to make the transdermal delivery system of the disclosure in patch form known to those skilled in the art, for example, such as but not limited to reservoir patch, matrix patch, drug in adhesives, film forming formulation, micro-dosing transdermal patch, transdermal films and may include, such as but are not limited to polymers, copolymers, derivatives, backing film, release membranes, release liners, etc. either alone or in combinations thereof. Pressure sensitive adhesives (such as but not limited to silicone polymers, rubber based adhesives, acrylic polymers, acrylic copolymers, polyisobutylene, acrylic acid-isooctyl acrylate copolymer, hot melt adhesives, polybutylene etc.), backing film (such as but not limited to ethylene vinyl acetate copolymers, vinyl acetate resins, polyurethane, polyvinyl chloride, metal foils, polyester, aluminized films, polyethylene, etc.), release membrane (such as but not limited to microporous polyethylene membrane, microporous polypropylene membrane, rate controlling ethylene vinyl acetate copolymer membrane etc.), release liners (such as but not limited to siliconized polyester films, fluoropolymer coated polyester film, polyester film, siliconized polyethylene terephthalate film, etc.), tapes, etc.

The transdermal formulation and/or topical formulation and/or transdermal delivery system of the disclosure may deliver at least therapeutic effective dose of active agent, THC and/or CBD, and its derivatives, alone or in combinations thereof in human plasma required for treating and/or preventing multiple sclerosis pain. Therapeutically effective active agent THC and/or CBD, and/or its derivatives dosages refers to the therapeutic concentration of in human plasma required for treating and/or preventing multiple sclerosis pain. Furthermore, the precise therapeutic effective dose of THC and/or CBD, and its derivatives in the transdermal formulation or topical formulation or transdermal delivery system can be determined by those skilled in the art based on factors such as but not limited to the patient's condition etc. The transdermal formulation or topical formulation or transdermal delivery system will be available in different dosage strengths and patch sizes in order to achieve optimum therapeutic outcome based on patient's requirement.

In yet another embodiment, the transdermal formulation and/or topical formulation and/or transdermal delivery system of the disclosure may deliver at least therapeutic effective dose of THC and/or CBD and derivatives of its. Therapeutically effective doses of active agent THC and/or CBD, and its derivatives refers to the therapeutic concentration of active agent in human plasma required for the treatment and/or prevention and/or control of multiple sclerosis pain.

The transdermal formulation or transdermal patch of active agent THC and/or CBD and its derivatives can be applied to the skin surface in any of the following dosage regimens such as once in a day, once in two days, once in three days, once in four days, once in five days, once in six days, once in a week, once in a range of from about 8 to about 13 days, once in two weeks, or once in 15 days.

### Example 12

### Example Formulations of Drug in Adhesive Matrix Patch

| Component | % W/W |
|---|---|
| Active component (THC and/or CBD) | 0.5%-30% |
| Solvent | 2%-30% |
| Permeation enhancer | 2%-30% |
| Pressure sensitive adhesive polymer | 20%-80% |
| Polymer | 1%-10% |

### Example Formulations of Drug in Adhesive Matrix Patch

| Component | % W/W |
|---|---|
| Active component (THC and/or CBD) | 1%-30% |
| Solvent | 2%-30% |
| Permeation enhancer | 2%-30% |
| Pressure sensitive adhesive polymer | 20%-80% |

### Example 13

Synthetic delta-9-thc (THC) and cannabidiol (CBD) formulations for transdermal delivery ((Formulation Nos. 009, 010, 011, 012, and 013) were prepared by mixing ingredients as shown in Table 1:

TABLE 1
Transdermal Synthetic Cannabidiol formulations

| | 001 | 002 | 003 | 004 | 005 |
|---|---|---|---|---|---|
| Ingredients | (% W/W) | (% W/W) | (% W/W) | (% W/W) | (% W/W) |
| CBD | 8.0 | 8.0 | 7.7 | 6.9 | 6.4 |
| THC | 8.0 | 8.0 | 7.7 | 6.9 | 6.4 |
| Ethanol | 42.7 | 37.3 | 37.0 | 33.0 | 25.4 |
| Propylene | 40.0 | 40.0 | 37.0 | 33.0 | 30.5 |
| Glycol | | | | | |
| Isopropyl | | | 4.7 | — | — |
| Palmitate | | | | | |
| DMSO | | | | 13.8 | 12.7 |
| Oleic Acid | | 5.3 | 4.7 | 5.5 | 5.1 |
| NMP | | | | — | 12.7 |

Abbreviations: THCH = delta-9-THC; CBD = Cannabidiol; NMP: N-methyl Pyrrolidone.

All of the components from Table 1, with the exception of the CBD and THC, were mixed together with stirring for 18 hours. Next, the CBD and THC were added into the excipient mixture to prepare the final transdermal formulations.

The prepared transdermal formulations were then subjected to a flux measurement test as follows. Human cadaver skin, stored at −80° C., was thawed at room temperature in phosphate buffered saline (PBS), and visually inspected for defects before using in the study. Transdermal flux was then measured using standard Franz diffusion cells composed of a cylindrical donor compartment and a separate water jacketed cylindrical receptor compartment with the volume of 13 mL. The human cadaver skin was clamped between the two compartments with the dermis side facing toward the receptor compartment. The donor compartment was filled with the transdermal CBD and THC formulations prepared as described above. The receptor compartment was filled with receptor medium, held at constant temperature, and constantly stirred to collect the CBD and THC as it diffuses through the skin and into receptor compartment. It is important to confirm that the receptor fluid is always in contact with the skin. The receptor compartment was emptied at 24 hr intervals for assay of CBD and THC and replaced with fresh receptor solution. In order to maintain the sink condition in receptor compartment, it is important to keep the CBD and THC concentration in receptor compartment less than 10% of its solubility. The experimental conditions are provided in Table 2:

TABLE 2
Experimental Condition for In-vitro Permeability testing

| | |
|---|---|
| Receiving Media | De-ionized water + 0.5% |
| | Brij-O(20) + 0.01% Sodium Azide |
| Receiving Media Volume (mL) | 13 |
| Sample Volume (mL) | 13 |
| Sampling Interval (hr) | 24, 48, 72 |
| Franz-cell diffusion area (sqcm) | 1.76 |
| Membrane Type | Human Cadaver Skin |

Flux of CBD and THC through the human cadaver skin was measured for a minimum period of 72 Hrs (3 days) and results of the flux measurement are provided in Table 3 and 4.

TABLE 3
CBD Flux Results

| | 009 | 010 | 011 | 012 | 013 |
|---|---|---|---|---|---|
| Average Flux | 0.53 | 0.86 | 1.10 | 1.39 | 1.01 |
| (0-24 hr) | (23.6%) | (27.0%) | (20%) | (19.9%) | (5.1%) |
| (ug/sqcm/hr) | | | | | |
| Average Flux | 0.92 | 1.16% | 1.21 | 1.44 | 1.21 |
| (24-48 hr) | (9.6%) | (14.4%) | (18.3%) | (1.65%) | (27.3%) |
| (ug/sqcm/hr) | | | | | |
| Average Flux | 0.52 | 0.86 | 0.71 | 1.27 | 1.01 |
| (48-72 hr) | (2.44%) | (12.9%) | (13%) | (16.3%) | (27.5%) |
| (ug/sqcm/hr) | | | | | |
| Average Flux | 0.66 | 0.96 | 1.01 | 1.37 | 1.08 |
| (0-72 hr) | | | | | |
| (ug/sqcm/hr) | | | | | |

TABLE 4
THC Flux Results

| | 009 | 010 | 011 | 012 | 013 |
|---|---|---|---|---|---|
| Average Flux | 0.0 | 0.28 | 0.62 | 0.73 | 0.60 |
| (0-24 hr) | | (89%) | (20.4%) | (24.5%) | (5.1%) |
| (ug/sqcm/hr) | | | | | |
| Average Flux | 0.39 | 0.65 | 0.78 | 0.85 | 0.80 |
| (24-48 hr) | (87%) | (20.8%) | (19.4%) | (8.7%) | (32.3%) |
| (ug/sqcm/hr) | | | | | |
| Average Flux | 0.0 | 0.47 | 0.43 | 0.73 | 0.58 |
| (48-72 hr) | | (20.3%) | (32%) | (20.2%) | (40%) |
| (ug/sqcm/hr) | | | | | |
| Average Flux | 0.13 | 0.47 | 0.61 | 0.77 | 0.66 |
| (0-72 hr) | | | | | |
| (ug/sqcm/hr) | | | | | |

### REFERENCE

1. Furgiuele A., et.al., “Immunomodulatory Potential of Cannabidiol in Mulitple Sclerosis: A systematic Review” J Neuroimmune Pharmacol, 2021, 1-19.
2. Dobson R., et.al., “Multiple Sclerosis-A review”, Eur J Neurol, 26, 27-40
3. Reich D S, et.al., “Multiple Sclerosis”, N Engl J Med, 378, 169-180
4. Oh J., et.al., “Multiple Sclerosis: Clinical Aspect”, Curr. Opin Neurol, 31, 752-759
5. Thompson A J, et.al., “Multiple Sclerosis”, Lancet, 391, 1622-1636
6. Yamout B I, et.al., “Multiple Sclerosis”, Semin Nurol, 38, 212-225
7. Hemmer B., et.al., “New Concenpt in the immunopathogenesis of multiple sclerosis”, Nat Rev Neurosci, 3, 291-301
8. Gholamzad M., et.al., “A comprehensive review on the treatment approaches of multiple sclerosis: Currently and in the future”, Inflamm Res, 68, 25-38
9. Yadav V., et.al., “Summary of evidence-based guideline: Complementary and alternative medicine in multiple sclerosis: report of the guideline development submcomitte of the American Academy of Neurology” Neurology, 82, 1083-1092
10. Schabas A J, et.al., “Cannabis-based product use in multiple sclerosis cohort”, Mult Scler J Exp Transl Clin, 5, 2055217319869360
11. Klein T W, “Cannabinoid-based drugs as anti-inflammatory therapeutics”, Nat Rev Immunol, 5, 400-411
12. Esposito G., et.al., “Cannabidiol in inflammatory bowel disease: a brief overview”, Phytother Res, 27, 633-636
13. Lowin T, et.al., “Joints for Joints: Cannabinoids in the treatment of rheumatoid arthritis”, Curr Opin Rhematol, 31, 271-278
14. Cassano T., et.al., “From Cannabis Sativa to Cannabidiol: promising therapeutics candidate for the treatment of neurodegenerative disease”, Front Pharmacol, 11, 124
15. Costiniuk C T., et.al., “Acute Inflammation and pathogenesis of SARS-COV-2 infection: Cannabidiol as a potential anti inflamattory treatment?” Cytokine Growth Factor Rev, 53, 63-65

While the invention has been described in detail and with reference to specific examples thereof, it will be apparent to one skilled in the art that various changes and modifications can be made therein without departing from the spirit and scope thereof.

What is claimed is:

1. A method for the treatment and/or prevention and/or control of chronic pain in a patient comprising:
selecting a patient in need of treatment and/or prevention and/or control of chronic pain;
topically applying a pharmaceutical composition in the form of a transdermal matrix patch comprising:
synthetic delta-9 tetrahydrocannabinol (THC), wherein the concentration of the synthetic delta-9 THC in the pharmaceutical composition is selected from the group consisting of about 0.5%, about 1%, about 2%, about 3%, about 4%, about 5%, about 6%, about 7%, about 8%, about 9%, about 10%, about 11%, about 12%, about 13%, about 14%, and about 15% w/w, and/or
synthetic cannabidiol (CBD), wherein the concentration of the synthetic CBD in the pharmaceutical composition is selected from the group consisting of about 0.5%, about 1%, about 2%, about 3%, about 4%, about 5%, about 6%, about 7%, about 8%, about 9%, about 10%, about 11%, about 12%, about 13%, about 14%, and about 15% w/w,
wherein the pharmaceutical composition further comprises:
about 5% to about 15% w/w of a solvent comprising propylene glycol;
about 6% to about 15% w/w of a penetration enhancer comprising oleic acid;
about 50% to about 80% w/w of a silicone pressure sensitive adhesive;
about 5% to about 15% w/w of at least one suspending agent comprising silicon dioxide;
about 0.1%, 0.2%, 0.3%, 0.4%, 0.5% or 0.6% of an antioxidant comprising BHT, wherein the transdermal matrix patch provides an average flux of the synthetic delta-9 THC of about 0.47 to about 0.85 μg/cm²/hr over at least 7 days, thereby treating, preventing and/or controlling chronic pain in the patient.

2. The method of claim 1 wherein the chronic pain is selected from the group consisting of neuropathic pain, peripheral neuropathic pain, inflammatory pain, musculoskeletal pain, pain due to muscle spasms, pain due to increased muscle tone, osteoarthritic pain, muscular headache, tension-type headache, migraine, cluster headache, atypical facial pain, referred pain, vulvodynia, proctodynia, and any combination thereof.

3. The method of claim 1 wherein the topical application of the transdermal pharmaceutical composition is for the treatment and/or prevention and/or control of chronic pain in a patient, and wherein the transdermal patch is applied at a time period selected from the group consisting of once in a day, once in two days, once in three days, once in four days, once in five days, once in six days, once in a week, and once in ten days, and once in fifteen days.

4. The method of claim 1 further providing a steady absorption rates of the active components of the transdermal patch over a time period selected from the group consisting of once in a day, once in two days, once in three days, once in four days, once in five days, once in six days, once in a week, once in ten days, and once in fifteen days.

5. The method of claim 1 further achieving a constant blood serum levels of the active components of the transdermal patch over a time period selected from the group consisting of once in a day, once in two days, once in three days, once in four days, once in five days, once in six days, once in a week, once in ten days, and once in fifteen days.

6. The method of claim 1 further achieving a reduced variability in dosage of the active components of the transdermal patches over a time period selected from the group consisting of once in a day, once in two days, once in three days, once in four days, once in five days, once in six days, once in a week, once in ten days, and once in fifteen days.

7. The method of claim 1 further providing a plasma concentration of the active components of the transdermal patch in a therapeutic range about 0.01 ng/mL to about 500 ng/mL.

8. The method of claim 1 further providing a plasma concentration of the active components of the transdermal patch in a therapeutic range of about 0.1 ng/ml to about 300 ng/mL.

9. A method for the treatment and/or prevention and/or control of multiple sclerosis in a patient comprising:
selecting a patient in need of treatment and/or prevention and/or control of multiple sclerosis;
topically applying a pharmaceutical composition in the form of a transdermal matrix patch comprising:
synthetic delta-9 tetrahydrocannabinol (THC), wherein the concentration of the synthetic delta-9 THC in the pharmaceutical composition is selected from the group consisting of about 0.5%, about 1%, about 2%, about 3%, about 4%, about 5%, about 6%, about 7%, about 8%, about 9%, about 10%, about 11%, about 12%, about 13%, about 14%, and about 15% w/w, and/or
synthetic cannabidiol (CBD), wherein the concentration of the synthetic CBD in the pharmaceutical composition is selected from the group consisting of about 0.5%, about 1%, about 2%, about 3%, about 4%, about 5%, about 6%, about 7%, about 8%, about 9%, about 10%, about 11%, about 12%, about 13%, about 14%, and about 15% w/w,
wherein the pharmaceutical composition further comprises:
about 5% to about 15% w/w of a solvent comprising propylene glycol;
about 6% to about 15% w/w of a penetration enhancer comprising oleic acid;
about 50% to about 80% w/w of a silicone pressure sensitive adhesive;
about 5% to about 15% w/w of at least one suspending agent comprising silicon dioxide;
about 0.1%, 0.2%, 0.3%, 0.4%, 0.5% or 0.6% of an antioxidant comprising BHT, wherein the transdermal matrix patch provides an average flux of the synthetic delta-9 THC of about 0.47 to about 0.85 μg/cm²/hr over at least 7 days,
thereby treating, preventing and/or controlling multiple sclerosis in the patient.

10. The method of claim 9 further providing a constant rate of delivery of the active components of the transdermal patch over a time period selected from the group consisting of once in a day, once in two days, once in three days, once in four days, once in five days, once in six days, once in a week, once in ten days, and once in fifteen days.

11. The method of claim 9 further providing a steady absorption rates of the active components of the transdermal patch over a time period selected from the group consisting of once in a day, once in two days, once in three days, once in four days, once in five days, once in six days, once in a week, once in ten days, and once in fifteen days.

12. The method of claim 9 further achieving a constant blood serum levels of the active components of the transdermal patch over a time period selected from the group consisting of once in a day, once in two days, once in three days, once in four days, once in five days, once in six days, once in a week, once in ten days, and once in fifteen days.

13. The method of claim 9 further achieving a reduced variability in dosage of the active components of the transdermal patches over a time period selected from the group consisting of once in a day, once in two days, once in three days, once in four days, once in five days, once in six days, once in a week, once in ten days, and once in fifteen days.

14. The method of claim 9 further providing a plasma concentration of the active components of the transdermal patch in a therapeutic range about 0.01 ng/ml to about 500 ng/mL.

15. The method of claim 9 further providing a plasma concentration of the active components of the transdermal patch in a therapeutic range of about 0.1 ng/ml to about 300 ng/mL.
